(19) United States
(12) Patent Application Publication
(10) Pub. No.: US 2024/0262927 A1
(43) Pub. Date: Aug. 8, 2024

(54) DIAGNOSTIC INDICATORS AND THERAPEUTIC TARGETS OF RHEUMATOID ARTHRITIS

(71) Applicant: KSL BIOMEDICAL, INC., Cheektowaga, NY (US)

(72) Inventors: Fanlei HU, Beijing (CN); Zhanguo LI, Beijing (CN)

(21) Appl. No.: 18/163,547

(22) Filed: Feb. 2, 2023

(51) Int. Cl. C07K 16/28; A61K 39/395; A61K 45/06; A61P 19/02; G01N 33/564

(52) U.S. Cl. CPC C07K 16/2896; A61K 39/3955; A61K 45/06; A61P 19/02; G01N 33/564; C07K 2317/73; C07K 2317/76; G01N 2800/102

(57) ABSTRACT

Rheumatoid arthritis (PA) is an autoimmune disease characterized by proliferative synovitis with deterioration of cartilage and bone. Osteoclasts (OCs) are the active participants in the bone destruction of RA. Many current therapeutic strategies for RA have limited effects on bone destruction. Macrophage scavenger receptor A (SR-A) is a class of pattern recognition receptors (PRRs) involved in bone metabolism and OC differentiation. Measuring the amount of SR-A is an effective tool to diagnose RA since the SR-A levels in a liquid biological sample were selectively elevated and positively correlated with bone destruction in patients with PA. Once a patient is diagnosed with RA, the patient with RA is administered with anti-SR-A neutralizing antibodies in order to (a) inhibit OC differentiation and bone absorption in the patient with RA (but not in healthy individuals); and (b) therapeutically decrease bone destruction in the patient with RA.

### FIELD OF THE INVENTION

Treating some adverse effects of rheumatoid arthritis.

### BACKGROUND OF THE INVENTION

Rheumatoid arthritis (PA) is a chronic, inflammatory autoimmune disease with the typical characteristic of periarticular bone erosion. The inflammation can lead to synovium proliferation, bone destruction, deformity, and function decline. In 1971, Sharp et al. proposed a system including the observation and scoring of hands and wrists in patients with RA to assess structural change. Van Der Heijde further modified the Sharp score for a better description of bone destruction, including bone erosion score and joint space narrowing (JSN) score. Bone erosion is a critical outcome indicator in RA, predicting a more severe course of disease with a higher degree of disability.

Osteoclasts (OCs) are the main active participants in RA bone destruction. They are multinucleated cells derived from hematopoietic precursors of the myeloid lineage with the potential of bone resorption. In RA, the generation, differentiation, and activation of OCs are increased as induced by proinflammatory cytokines such as tumor necrosis factor alpha (TNF-α) and interleukin 6 (IL-6), and autoantibodies such as anti-citrullinated protein antibodies (ACPA) and rheumatoid factor (RF), resulting in bone resorption and destruction. Therefore, inhibiting the formation and functions of OCs are ideal and effective approaches for RA targeted therapy. Although several inhibitors have been developed, including bisphosphonates, denosumab, and natural compounds such as xylitol, till now there are few therapeutic drugs targeting OCs available. Current drugs for RA treatment mainly consist of nonsteroidal anti-inflammatory drugs (NSAIDs), glucocorticoids (GC), and disease-modifying antirheumatic drugs (DMARDs) including conventional DMARDs (cDMARDs), biological DMARDs (bDMARDs), and targeted synthetic DMARDs (tsDMARDs). Although with great advances, their effects on inhibiting OCs and preventing joint and bone destruction are insufficient with limitations.

Macrophage scavenger receptor A (SR-A, CD204, MSR-1, SCARA1) is a kind of classical pattern recognition receptors (PRRs) primarily expressed on macrophages. Macrophage scavenger receptor A has also been indicated to have a critical involvement in T-cell immune responses and B-cell immune responses. Besides cancer, cardiovascular disease, and Alzheimer's disease, the role of SR-A in autoimmune diseases, such as systemic lupus erythematosus, autoimmune hepatitis, and inflammatory bowel diseases have also been recognized. Moreover, compared to wild-type (WT) mice, SR-A+ mice revealed increased bone mass, bone thickness, bone density, and trabeculae number, which suggested the role of SR-A in bone metabolism and OC differentiation. SR-A was then demonstrated to be able to directly promote OC differentiation. SR-A may also activate the extracellular signal-regulated kinases (ERK) and the c-Jun N-terminal kinases (JNK) signaling pathways and increase IL-6 production to further stimulate the formation of OCs. In a prior large-scale, multicenter study, it was unveiled that a significant elevation of soluble SR-A (sSR-A) in the patient serum with RA, serving as a potential diagnostic marker. It was further revealed that SR-A−/− mice were resistant to collagen-induced arthritis (CIA). Administration of SR-A recombinant protein exacerbated the incidence and progression of CIA, while SR-A neutralizing antibody ameliorated the severity of arthritis. However, the therapeutic potential of SR-A blockade against bone destruction remained to be elucidated.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-D are correlation analyses of the level of sSR-A with bone destruction in patients with RA. FIG. 1A illustrates that the serum—a liquid biological sample—level of sSR-A was higher in patients with RA than that in healthy individuals (PA, n=80; healthy control (HC), n=60; ***P<0.001), wherein the results were presented as mean±SEM two-tailed Mann-Whitney U test. FIG. 1B illustrates the level of sSR-A in patients with RA was positively correlated with the SHARP score (r=0.2308, *P=0.0395). FIG. 1C illustrates the level of sSR-A in patients with RA was positively correlated with the erosion score (r=0.2358, *P=0.0352). FIG. 1D illustrates the level of sSR-A in patients with RA was positively correlated with and the joint space narrowing score (r=0.2277, *P=0.0422). The results for FIGS. 1B, 1C, and 1D were presented as mean±SEM two-tailed Spearman's rank correlation test.

FIGS. 2A and 2B illustrates the potential involvement of sSR-A in RA bone destruction. Monocytes from healthy individuals (n=6) were co-cultured with the serum of the patient with RA pre-incubated with 2 μg/ml anti-human SR-A neutralizing antibody or isotype antibody, 100 ng/ml RANKL, and 50 ng/ml M-CSF. After 14 days, the cells were fixed and set for TRAP staining, and multinucleated cells (more than three nuclei) with TRAP positivity were counted. The representative figures are illustrated in FIG. 2A and the statistical result were illustrated at FIG. 2B, wherein a two-tailed Wilcoxon matched-paired signed-rank test, *P<0.05) was used and the scale bar=50 μm.

FIGS. 3A, 3b, 3C, 3D, and 3E illustrate SR-A neutralizing antibody inhibits OC differentiation and bone destruction in patients with RA. FIG. 3A illustrates monocytes from patients with RA (n=6) that were co-cultured with isotype antibody or anti-human SR-A neutralizing antibody at 2 μg/ml for 14 days in the presence of 100 ng/ml receptor activator of nuclear factor kappa-B ligand (RANKL) and 50 ng/ml macrophage colony-stimulating factor (M-CSF). Then the cells were fixed and set for tartrate-resistant acid phosphatase (TRAP) staining, and multinucleated cells (more than three nuclei) with TRAP positivity were counted. The representative figures and the statistical result were shown, respectively. Scale bar=50 μm.

FIG. 3B illustrates the SR-A neutralizing antibody showed minimal effects on OC differentiation in healthy individual monocytes. OC differentiation was performed as described above (n=6), and the representative figures and statistical results were shown, respectively. Scale bar=50 μm. FIG. 3C illustrates SR-A neutralizing antibody inhibited bone destruction in patients with RA. OC differentiation was performed as describe above with the bone slices at the bottom of the 96-well plates (n=6). Then the resorption area was stained and analyzed. FIG. 3D illustrates the differentiated OCs from patients with RA as described in FIG. 3A that were set for qPCR analysis of the expression of the OC maker genes, including CTSK, TRAP, and MMP-9. FIG. 3E illustrates the level of IL-6 in the cell culture supernatants of patients with RA was also detected by ELISA. The statistical results are presented as mean±SEM (two-tailed Wilcoxon matched-paired signed-rank test, *P<0.05, **P<0.01, ns, not significant).

FIGS. 4A and 4B illustrate SR-A neutralizing antibody dampens OC differentiation in collagen-induced arthritis (CIA) mice in vitro. Bone marrow-derived monocytes (BMMs) from CIA mice are illustrated in FIG. 4A, n=6; and naïve mice are illustrated in FIG. 4B, n=6 were co-cultured with isotype antibody or anti-mouse SR-A neutralizing antibody at 2 μg/ml in the presence of 100 ng/ml RANKL and 50 ng/ml M-CSF. After 9 days, the cells were fixed for TRAP staining. The typical figures and the statistical results were shown, respectively (two-tailed paired Student's t-test, *P<0.05, ns, not significant). Scale bar=50 μm.

FIGS. 5A, 5B, 5C, and 5D illustrate SR-A neutralizing antibody ameliorates osteoclastogenesis in CIA mice in vivo. After the initiation of disease, CIA mice with similar scores of 2-3 were injected with anti-mouse SR-A neutralizing antibody or isotype antibody (2 μg/mouse) intravenously every 2 days for a total of five injections (n=5 per group). FIG. 5A illustrates that after the sacrifice, the paws from SR-A neutralizing antibody, or isotype antibody-treated CIA mice, or untreated CIA mice, as well as naïve mice were fixed, paraffin-embedded, sectioned, stained with TRAP, and analyzed by NDP.view2. Arrows indicate osteoclastogenesis. Scale bar=100 μm. FIG. 5B illustrates micro-computed tomography (CT) imaging of paws from different group mice as in FIG. 5A was also performed. Arrows indicate the bone erosion and destruction. FIGS. 5C and 5D illustrate the serum levels of IL-6 (FIG. 5C) and C-terminal telopeptide (FIG. 5D) in different group mice were detected by ELISA. The statistical results are presented as mean±SEM (One-way analysis of variance (ANOVA) test followed by Dunnett's posttest for multiple comparisons, *P<0.05, ***P<0.001, ns, not significant).

FIGS. 6A and 6B illustrate SR-A neutralizing antibody attenuates osteoclastogenesis in CIA mice ex vivo. Bone marrow-derived monocytes (BMMs) from CIA mice treated with SR-A neutralizing antibody or isotype antibody as described in FIGS. 5A-5D were harvested for osteoclast differentiation ex vivo. After 9 days, the cells were fixed and set for TRAP staining, and multinucleated cells (more than three nuclei) with TRAP positivity were counted. The typical figures and the statistical result were shown, respectively in FIG. 6A. Scale bar=50 μm. The relative expression of CTSK, TRAP, and MMP-9 was also evaluated by qPCR in FIG. 6B. The statistical results are presented as mean±SEM (two-tailed paired Student's t test, *P<0.05, **P<0.01).

FIGS. 7A, 7B, 7C, and 7D illustrate the prevalence of sSR-A in patients with RA. FIGS. 7A, 7B, 7C illustrate the serum levels of sSR-A were significantly higher in patients with RA than those of healthy individuals and patients with other common rheumatic diseases and non-autoimmune inflammatory diseases. FIG. 7a illustrates patients with other common rheumatic diseases systemic lupus erythematosus (SLE), (***p=1.1346E-10), Sjogren's Syndrome (SS), (***p<1E-15), ankylosing spondylitis (AS), (***p=1.1289E-08), Gout (**p=0.0035), psoriatic arthritis (PsA), (***p=1.3426E-04), ANCA-associated vasculitis (AAV, ***p=2.0262E-07), adult onset still's disease (AOSD), (***p=2.98E-13), polymyalgia rheumatic (PMR), (***p=8.2141E-06), and osteoarthritis (OA), (***p=2E-15)) and healthy controls (***p=4E-15). FIG. 7B illustrates patients with autoimmune hepatitis (AIH), (***p=7.8003E-04) and healthy controls (***p<1E-15). FIG. 7C shows patients with non-autoimmune inflammatory diseases (NAID), including enteritis, gastritis, pneumonia, and colitis, (***p<1E-15) and healthy controls (***p=3.8E-14). FIG. 7D conveys sSR-A were higher in systemic lupus erythematosus (SLE) or Sjogren's syndrome (SS) overlapped with RA patients than SLE overlapped with SS patients (***p=9.8292E-10), SLE patients (***p=3.1014E-07) and SS patients (***p=1.9579E-11). RA=107; SLE=30; SS=30; ankylosing spondylitis (AS)=39; Gout=39; psoriatic arthritis (PsA)=39; ANCA-associated vasculitis (AAV)=39; adult-onset Still's disease (AOSD)=30; polymyalgia rheumatica (PMR)=24; osteoarthritis (OA)=20; healthy controls (HC)=90; autoimmune hepatitis (AIH)=25; NOD2-associated autoinflammatory disease (NAID)=71; SLE or SS overlapped with RA=98; SLE overlapped with SS=31. Red horizontal lines: means; error bars: SEMs. **p<0.01, ***p<0.001 (Kruskal-Wallis test followed by Dunn's posttest for multiple comparisons).

FIGS. 8A, 8B, 8C, and 8D illustrate the results of the detection of sSR-A in a large-scale, multicenter study. That large-scale, multicenter study was performed to assess the discriminative power of serum sSR-A for RA using quantitative ELISA. The results of the training cohort, validation cohort 1, validation cohort 2, and the pooled three cohorts were shown, respectively. FIG. 8A illustrates the Beijing cohort: training cohort (PA=528; SLE=254; SS=120; OA=72; HC=480; ***all p<1E-15), FIG. 8B illustrates the Inner Mongolia cohort: validation cohort 1 (RA=213; SLE=144; SS=144; QA=119; HC=120; ***all p<1E-15), FIG. 8C illustrates the Hangzhou cohort: validation cohort 2 (RA=155; SLE=80; SS=55; QA=32; HC=100. ***p<1E-15, =2.1799E-04, =5.9038E-05, and <1E-15, respectively, from left to right), FIG. 8D illustrates the pooled three cohorts: pooled all three cohorts' data together (***all p<1E-15). Red horizontal lines: means; error bars: SEMs. ***p<0.001 (Kruskal-Wallis test followed by Dunn's posttest for multiple comparisons).

FIGS. 9A, 9B, 9C, and 9D illustrate AROC curves of sSR-A for RA diagnosis. Covariate-adjusted receiver operating characteristic curve (AROC) analysis was performed to evaluate the performance of sSR-A in diagnosis of RA, with age and gender as the confounders. The area under the age-adjusted and gender-adjusted ROC curve (AAUC) was 0.8420 (95% CI extending from 0.8094 to 0.8688) in Beijing cohort (see, FIG. 9A); 0.8641 (95% CI extending from 0.8232 to 0.9013) in Inner Mongolia cohort (see, FIG. 9B), 0.8219 (95% CI extending from 0.7502 to 0.8748) in Hangzhou cohort (see. FIG. 9C), and 0.8436 (95% CI extending from 0.8225 to 0.8669) in the Pooled three cohorts (see, FIG. 9D). AROC: covariate-adjusted receiver operating characteristic curve; AAUC: area under the covariate-adjusted ROC curve; CI: confidence interval (covariate-adjusted ROC curve analysis).

FIGS. 9E and 9F are AROC curves of SR-A combined with anti-CCP for RA diagnosis (FIG. 9E) and ERA diagnosis (FIG. 9F). FIG. 9E illustrates a covariate-adjusted receiver operating characteristic curve (AROC) analysis was performed to evaluate the performance of SR-A combined with anti-CCP for the diagnosis of RA, with age and gender as the confounders. The area under the age-adjusted and gender-adjusted ROC curve (AAUC) was 0.8167 for SR-A, 0.8954 for anti-CCP, 0.9039 for the combination of SR-A and anti-CCP. While FIG. 9F illustrates a covariate-adjusted receiver operating characteristic curve (AROC) analysis was performed to evaluate the performance of SR-A combined with anti-CCP for the diagnosis of ERA, with age and gender as the confounders. The area under the age-adjusted and gender-adjusted ROC curve (AAUC) was 0.8487 for SR-A, 0.9105 for anti-CCP, 0.9205 for the combination of SR-A and anti-CCP.

FIGS. 10A, 10B, 10C, and 10D illustrate data for sSR-A in RA patients with normal erythrocyte sedimentation rate (ESR) and/or c-reactive protein (CRP). RA patients from the training and validation cohorts as well as the pooled cohort were divided into the following four groups, and the levels of sSR-A as well as the positive rates were further analyzed: RA patients with normal ESR (−) and normal CRP (−), RA patients with normal ESR (−) and increased CRP (+), RA patients with increased ESR (+) and normal CRP (−), RA patients with increased ESR (+) and increased CRP (+). FIG. 10A is for the Beijing cohort: training cohort (***p<1E-15, =3.5748E-07, <1E-15, and <1E-15, respectively, from left to right), FIG. 10B is for the Inner Mongolia cohort: validation cohort 1 (***p=4.6610E-06, =6.7480E-11, and <1E-15, respectively, from left to right), FIG. 10C is for the Hangzhou cohort: validation cohort 2 (**p=0.0069, ***p=1.6946E-04, =1.2122E-10, and <1E-15, respectively, from left to right), FIG. 10D is for the pooled three cohorts (***p<1E-15, =5.6245E-10, <1E-15, and <1E-15, respectively, from left to right). n: the number of sSR-A-positive patients; N: the number of total patients. -: normal; +: increased. Red horizontal lines: means; error bars: SEMs. **p<0.01, ***p<0.001, NS, not significant (Kruskal-Wallis test followed by Dunn's posttest for multiple comparisons).

FIGS. 11A, 11B, 11C, and 11D illustrate graphs representing data regarding sSR-A in ERA, anti-cyclic citrullinated peptide (CCP) and/or RF-negative RA and UA patients. FIG. 11A illustrates the levels of sSR-A in early RA (EPA) patients, including patients with disease duration <24 months (n=251, ***p<1E-15), <12 months (n=178, ***p<1E-15), and <6 months (n=91, ***p<1E-15) were detected. The positive rates of sSR-A were also analyzed. FIG. 11B illustrates the graph for the levels of sSR-A as well as the positive rates in anti-CCP and/or RF-negative RA patients, including anti-CCP-RA patients (n=179, ***p<1E-15), RF-RA patients (n=276, ***p<1E-15), and (anti-CCP and RF)-RA patients (n=155, ***p<1E-15) were examined. FIG. 11C illustrates a graph that compares sSR-A levels and positive rates in UA (n=119, ***p<1E-15), EPA (n=251, ***p<1E-15) and RA (n=896, ***p<1E-15) patients. While FIG. 11D illustrates the prevalence of sSR-A, anti-CCP, and RF in undifferentiated arthritis (UA) patients. n: the number of sSR-A-positive patients; N: the number of total patients. Red horizontal lines: means; error bars: SEMs. ***p<0.001, NS, not significant (Kruskal-Wallis test followed by Dunn's posttest for multiple comparisons shown in FIGS. 11A, 11B, and 11C or Chi-square test in FIG. 11D).

FIGS. 12A-12H illustrate elevation of sSR-A accelerates arthritis onset and exacerbates disease severity in mice. FIG. 12A illustrates a graph for the serum levels of mouse sSR-A as examined in the collagen-induced arthritis (CIA) and control mice by ELISA. The sSR-A levels were significantly higher in CIA mice than in adjuvant immunized or untreated DBA/1 mice (Naïve, n=20; Adjuvant, n=9; CIA, n=36. *p=0.0422 (left) and 0.0343 (right)). FIG. 12B shows a scheme of the experimental setup. DBA/1 mice were intravenously injected with recombinant SR-A protein (5 μg/mouse) or Saline every 2 days, starting from 2 days before boosting immunization for a total of five times. Mice were monitored every 2 days and sacrificed 2 weeks after the second immunization. FIGS. 12C and 12D show the arthritis incidence and the clinical scores for both CIA-Saline and CIA-SR-A mice over time (CIA-SR-A, n=13; CIA-Saline, n=13, ***p=2.4276E-04). FIG. 12E shows micro-CT image showing the bone destruction of paws from Naïve, CIA-SR-A, and CIA-Saline mice. FIG. 12F show H&E staining of the sagittal sections of paws from Naïve, CIA-SR-A, and CIA-Saline mice. Arrows indicate the stenosis of articular cavity and destruction of cartilage. Scale bar=100 μm. FIG. 12G shows the serum IL-17A levels were examined in Naïve, CIA-SR-A, and CIA-Saline mice (n=5 per group, **p=0.0082, ***p=2.8143E-04). While FIG. 12H shows the frequency of Th17 cells in the spleen was analyzed by flow cytometry. The representative flow charts and the statistical results were shown (Naïve, n=4; CIA-SR-A, n=7; CIA-Saline, n=6, **p=0.0093, ***p=6.5803E-04). Data are presented as mean±SEM. Results are representative of three independent experiments. *p<0.05, **p<0.01, ***p<0.001, NS, not significant (one-way ANOVA test followed by Tukey's posttest for multiple comparisons of FIGS. 12A, 12G, and 12H or two-way repeated measures ANOVA test of FIG. 12D).

FIGS. 13A-13G illustrate reduced incidence of CIA and autoimmune inflammation in SR-A-deficient mice. FIG. 13A illustrates the serum levels of mouse sSR-A in naïve C57BL/6 mice or CIA mice with different clinical scores were examined by ELISA (n=3 per group, *p=0.0155, ***p=1.7785E-05). FIG. 13B illustrates male WT (n=5) and SR-A−/− (n=5) mice were immunized with 200 μg chicken collagen II emulsified in CFA. The arthritis incidence (*p=0.049) and disease severity (*p=0.0171) were followed. FIG. 13C illustrates representative gross image of arthritis symptoms was recorded at 6 weeks post immunization. Inflammatory cell infiltration and bone erosion were evaluated by H&E staining. Red star indicates inflammatory cell infiltration. Arrow indicates bone erosion. Scale bar=100 μm. FIG. 13D illustrates the infiltration of MDSCs and Th17 cells into the inflamed joints was determined by flow cytometry. FIG. 13E illustrates six weeks after CIA induction, serum level of IL-17A in the arthritic mice (n=3 per group, *p=0.0387) was determined by ELISA. FIGS. 13F and 13G illustrate splenocytes from naïve or CIAWT/SR-A−/− mice were cultured in the presence of denatured collagen (50 μg/mL) for 48 h. IL-17A levels in the culture medium were determined by ELISA (FIG. 13F, top, ***p=1.5376E-04). Frequencies of IL-17A-producing cells were evaluated by ELISPOT (FIG. 13F, bottom, **p=0.0085) or intracellular cytokine staining (FIG. 13G, top). The frequencies of TNF-α-producing CD4+ T cells upon collagen stimulation were also assayed by intracellular cytokine staining (FIG. 13G, bottom). Data are presented as mean±SEM. Results are representative of three independent experiments. *p<0.05, **p<0.01, ***p<0.001, NS, not significant (one-way ANOVA test followed by Dunnett's posttest for multiple comparisons FIG. 13A, LogRank test and two-way repeated measures ANOVA test FIG. 13B, or two-tailed Student's t test FIGS. 13E and 13F).

FIGS. 14A to 14G illustrate the inhibition of SR-A ameliorates severity of arthritis in mice. FIG. 14A illustrates the scheme of the experimental setup. Intervention was initiated when the arthritis scores were averagely over 2. CIA mice were intravenously injected with SR-A neutralizing antibody or isotype Immunoglobulin G (IgG) (2 μg/mouse) once every 2 days for seven doses, or were intraperitoneally injected with SR-A inhibitor Fucoidan (100 μg/mouse) on day 0 and day 6 after initiation for two doses. FIG. 14B illustrates the arthritis scores in each intervention group were recorded daily after the first treatment (n=5 per group, **p=0.0028 (top) and 0.0016 (bottom), respectively). FIG. 14C illustrates H&E staining of the sagittal sections of paws from the isotype antibody-, SR-A neutralizing antibody-, or Fucoidan-treated CIA mice and naïve mice. Arrows indicate the stenosis of articular cavity and destruction of cartilage. Scale bar=100 μm. FIG. 14D illustrates micro-CT image showing the bone destruction of paws from the isotype antibody-, SR-A neutralizing antibody-, or Fucoidan-treated CIA mice and naïve mice. FIG. 14E illustrates the serum IL-17A levels were detected in the isotype antibody-, SR-A neutralizing antibody-, or Fucoidan-treated CIA mice and naïve mice (n=5 per group, **p=0.0081, ***p=1.7201E-04 (left) and 2.9233E-04 (right)). FIG. 14F illustrates the heart body weight ratio was shown to reflect the heart functions (n=5 per group). FIG. 14G illustrates H&E staining of the sagittal sections of liver, spleen, kidney, and heart were performed to evaluate the organ damage. Scale bar=100 μm. Data are presented as mean±SEM. Results are representative of three independent experiments. **p<0.01, ***p<0.001, NS, not significant (two-way repeated measures ANOVA test FIG. 14B or One-way ANOVA test followed by Tukey's posttest for multiple comparisons FIGS. 14E and 14F).

### SUMMARY OF THE INVENTION

Rheumatoid arthritis (PA) is an autoimmune disease characterized by proliferative synovitis with deterioration of cartilage and bone. Osteoclasts (OCs) are the active participants in the bone destruction of RA. Many current therapeutic strategies for RA have limited effects on bone destruction. Macrophage scavenger receptor A (SR-A) is a class of pattern recognition receptors (PRRs) involved in bone metabolism and OC differentiation. Measuring the amount of SR-A is an effective tool to diagnose RA since the SR-A levels in a liquid biological sample are positively correlated with bone destruction in patients with PA. Once a patient is diagnosed with RA, the patient with RA is administered with anti-SR-A neutralizing antibodies in order to (a) inhibit OC differentiation and bone absorption in the patient with RA (but not in healthy individuals); and (b) therapeutically decrease bone destruction in the patient with RA.

### DETAILED DESCRIPTION OF THE INVENTION

This invention is aimed to characterize the correlation between sSR-A and bone destruction in a patient with RA and reveal the capacity of SR-A neutralizing antibody in inhibiting OC differentiation, to provide an approach for RA bone destruction therapy.

### Early Diagnosis

Early diagnosis is critical to improve outcomes in rheumatoid arthritis (PA), but current diagnostic tools have limited sensitivity. Serum levels of soluble scavenger receptor-A (sSR-A) are increased in patients with RA and correlate positively with clinical and immunological features of the disease. This discriminatory capacity of sSR-A is clinically valuable and complements the diagnosis for early-stage SA and seronegative RA. sSR-A also has 15.97% prevalence in undifferentiated arthritis patients. Furthermore, administration of SR-A accelerates the onset of experimental arthritis in mice, whereas inhibition of SR-A ameliorates the disease pathogenesis. Together, these data identify sSR-A as a potential biomarker in diagnosis of RA, and targeting SR-A is a therapeutic strategy.

Rheumatoid arthritis (PA) is a chronic autoimmune disease that can lead to joint destruction, disability, and premature mortality. An estimated 50% of RA patients become permanently work disabled within 2-3 years of diagnosis. It affects about 1% of the population worldwide and a large-scale survey of residents showed that the prevalence of RA in, at least, China is 0.28%7. Although early diagnosis of RA is often associated with better response to treatment, reduced co-morbidity, and lower mortality, the rate of disease remission is only 8.6%.

The current RA classification criteria proposed by ACR/EULAR in 2010 improves sensitivity for early detection of RA as compared to the former classification system proposed by ACR in 1987. The biomarkers, i.e., rheumatoid factor (RF) and anticyclic citrullinated peptide antibody (anti-CCP) used in the current classification criteria only show a modest discriminating power. The sensitivity and specificity are 67% and 95% for anti-CCP, and 69% and 85% for RF, respectively. Although several biomarkers have been identified in peripheral blood or synovial fluid of RA patients, none of those achieves better specificity and sensitivity than anti-CCP alone. Therefore, there remain unmet needs to develop additional diagnostic tools as well as treatment options for RA.

Scavenger receptor-A (SR-A), also termed CD204, is a pattern recognition receptor primarily expressed on the cells of myeloid origin and displays pleiotropic biological functions. SR-A was initially identified as a major receptor on macrophages for internalization of modified lipoproteins. A wealth of studies have described the roles of SR-A in lipid metabolism, cardiovascular diseases, and pathogen recognition. Prior research has established an immunoregulatory activity of SR-A in attenuating cancer vaccination-induced antitumor immune responses and in limiting T-cell activation in inflammatory hepatitis. Both cell-associated SR-A (cSR-A) and soluble SRA (sSR-A) exhibit T cell suppressive activity via functional regulation of innate immune cells, e.g., dendritic cells (DCs) and myeloid-derived suppressor cells.

The data from a large-scale, multicenter study was evaluated to assess the discriminative power of sSR-A in a liquid biological sample for RA diagnosis. An elevation of sSR-A is present exclusively in patients with RA, but not in patients with systemic lupus erythematosus (SLE), Sjogren's Syndrome (SS), osteoarthritis (OA), ankylosing spondylitis (AS), gout, psoriatic arthritis (PsA), ANCA-associated vasculitis (AAV), adult-onset still's disease (AOSD), polymyalgia rheumatica (PMR), autoimmune hepatitis (AIH) or non-autoimmune inflammatory diseases (NAID). sSR-A not only demonstrates potential discriminatory ability for RA, but also complements the diagnosis for early RA as well as for seronegative RA. Using mouse arthritis models, the evidence indicates that increasing the levels of SR-A accelerates arthritis progression whereas inhibition of SR-A ameliorates disease severity. These findings support diverse functions of SR-A under different pathological conditions, and provide diagnostic and therapeutic strategies for clinical management of RA.

The Prevalence of sSR-A in RA.

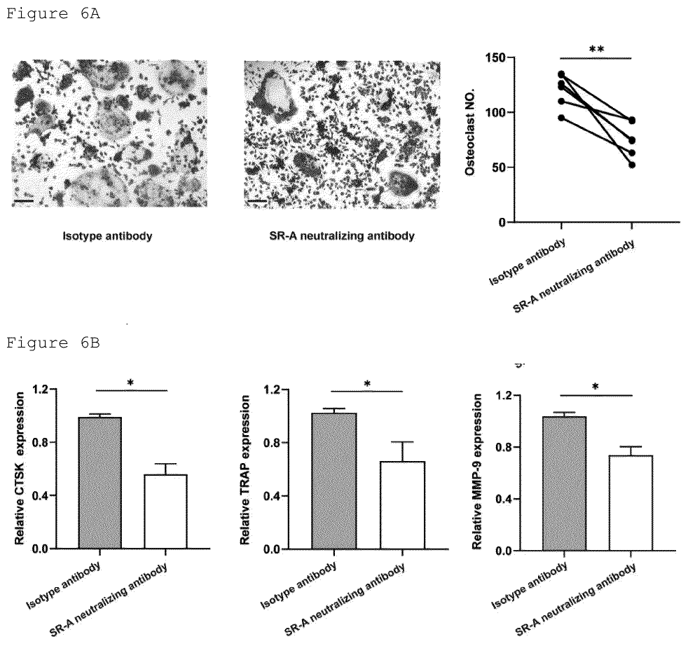

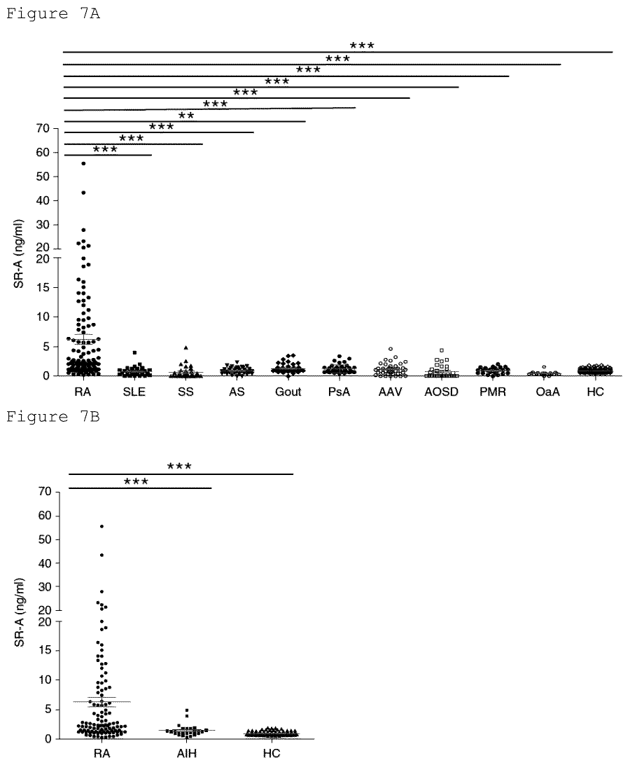

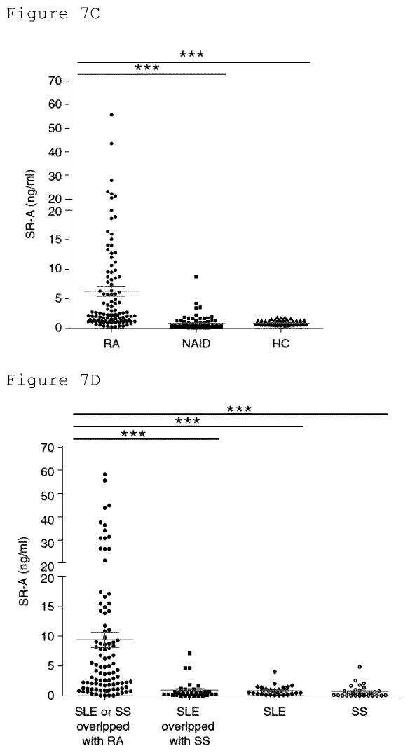

The levels of sSR-A in patients with RA or other types of rheumatic diseases and non-autoimmune inflammatory diseases were accessed. As shown in FIG. 7a, the serum levels of sSR-A in patients with RA were significantly higher than those of healthy individuals or patients with other common rheumatic diseases, including SLE, SS, OA, AS, Gout, PsA as well as AAV, AOSD, and PMR. Although elevation of sSR-A was previously reported in patients with hepatitis, the levels of sSR-A in RA patients were much higher compared to those in patients with autoimmune hepatitis (AIH, see, FIG. 7B). As illustrated in FIG. 7B, RA could be indicated—in other words a physician or equivalent thereof could identify as a differential diagnosis—in a subject when a measurement of SR-A in the subject's liquid biological sample—which could be serum, plasma, synovial fluid, or combinations thereof (notice this list does not include whole blood)—was 5 ng/ml or greater.

FIG. 7B also confirms that RA could be indicated—in other words a physician or equivalent thereof could identify as a differential diagnosis—in a subject when a measurement of SR-A in the subject's liquid biological sample—which could be serum, plasma, synovial fluid, or combinations thereof (notice this list does not include whole blood)—was greater than 7 ng/ml. Likewise, RA could be indicated—in other words a physician or equivalent thereof could identify as a differential diagnosis—in a subject when a measurement of sSR-A in the subject's liquid biological sample—which could be serum, plasma, synovial fluid, or combinations thereof—was 5 ng/ml or greater; and with greater certainty when the measurement of sSR-A in the subject's liquid biological sample—which could be serum, plasma, synovial fluid, or combinations thereof—was greater than 7 ng/ml. That was confirmed in the data that illustrates sSR-A was elevated in RA patients but not in patients with non-autoimmune inflammatory diseases (NAID), including enteritis, gastritis, pneumonia, and colitis (see, FIG. 7C). Strikingly, SLE or SS patients complicated with RA showed fundamentally increased levels of sSR-A compared with those without RA (see, FIG. 7D). All these results suggested that sSR-A was selectively elevated in RA patients.

The Diagnostic Value of sSR-A in RA.

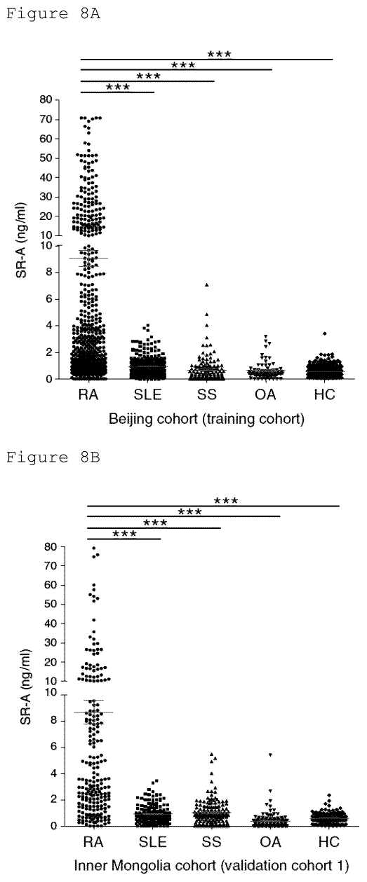

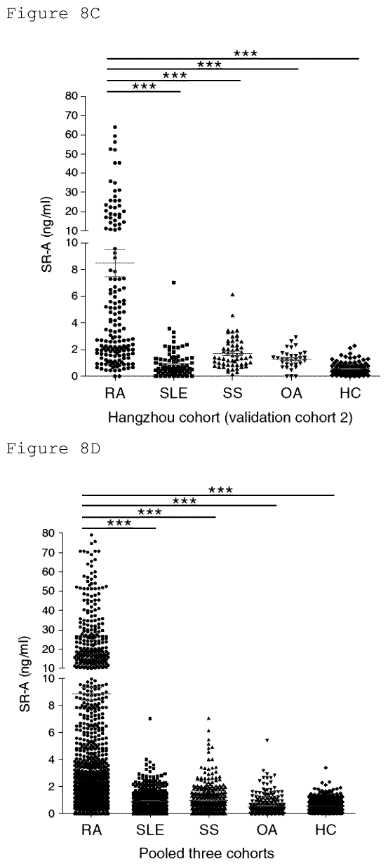

To determine the potential diagnostic value of serum sSR-A for RA, a large scale, multicenter study was conducted. A total of 2616 serum samples were collected, including 1454 samples in the Beijing cohort (training cohort), 740 and 422 samples in the Inner Mongolia and Hangzhou cohort, respectively (validation cohorts 1 and 2). This showed that the levels of sSR-A in RA patients within the training cohort were substantially higher than those of patients with other rheumatic diseases and healthy controls (median 2.77 ng/mL, mean 9.04 ng/mL, SD 14.24 ng/mL, p<0.001, see, FIG. 8A). The same results were also seen in the two validation cohorts (see, FIGS. 8B and 8C), which was further confirmed by the pooled data from the three cohorts (see, FIG. 8D).

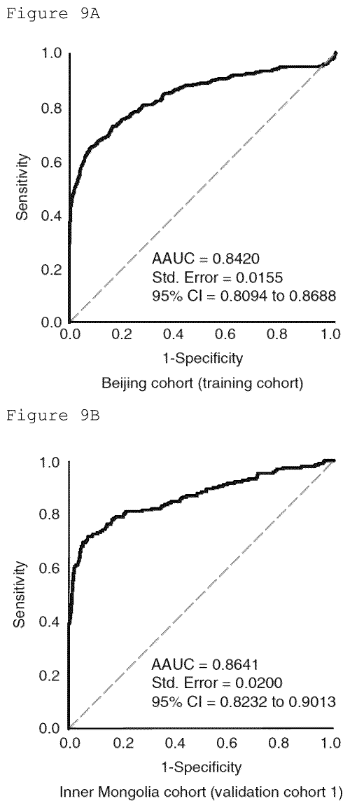

The covariate-adjusted receiver operating characteristic curve (AROC) analysis using non-parametric method was performed to evaluate the performance of sSR-A in diagnosis of RA, with age and gender as the confounders identified by the multivariable logistic regression analysis. The result revealed a significant area under the age-adjusted and gender-adjusted ROC curve (AAUC) of 0.8420 (95% CI extending from 0.8094 to 0.8688) for sSR-A in Beijing cohort (see, FIG. 9A). The AAUCs of Inner Mongolia and Hangzhou cohorts were 0.8641 (95% CI extending from 0.8232 to 0.9013) and 0.8219 (95% CI extending from 0.7502 to 0.8748), respectively (see, FIGS. 9A, 9B, and 9C). Pooling the data of the three cohorts yielded an AAUC of 0.8436 for sSR-A (see, FIG. 9D), approximate with that for anti-CCP (0.84) and RF (0.83) as reported. These results indicate that sSR-A reveals potential capacity in distinguishing RA.

The optimal cut-off value in the study was set for 3 standard deviations (SD) above the mean value of the healthy controls, which showed better clinical utility of sensitivity and specificity than the ROC curve and Youden index analysis. Based on the threshold value of 1.7024 ng/mL, the sensitivity and specificity of sSR-A for identification of RA were 61.36% and 94.38% in Beijing cohort, 73.24% and 90.51% in Inner Mongolia cohort, 74.19% and 83.15% in Hangzhou cohort, respectively. Analysis of the pooled three cohorts revealed the sensitivity of 66.41% and specificity of 91.45% for sSR-A in RA diagnosis, with PPV of 80.19% and NPV of 83.94% (Table 1). All these suggested that sSR-A demonstrated a diagnostic value slightly lower than anti-CCP, but higher than RF. The reported average sensitivity and specificity are 67% and 95% for anti-CCP, and 69% and 85% for RF, respectively.

TABLE 1
Sensitivity and specificity of sSR-A in RA diagnosis.

| Cohorts | Groups | N | Positive (n) | Sensitivity (%) | Specificity (%) | PPV (%) | NPV (%) |
|---|---|---|---|---|---|---|---|
| Beijing cohort (training cohort) | RA | 528 | 324 | 61.36 | 94.38 | 86.17 | 81.08 |
| | SLE | 254 | 31 | 12.20 | — | — | — |
| | SS | 120 | 11 | 9.17 | — | — | — |
| | OA | 72 | 5 | 6.94 | — | — | — |
| | HC | 480 | 5 | 1.04 | — | — | — |
| Inner Mongolia cohort (validation | RA | 213 | 156 | 73.24 | 90.51 | 75.73 | 89.33 |
| cohort 1) | SLE | 144 | 19 | 13.19 | — | — | — |
| | SS | 144 | 26 | 18.06 | — | — | — |
| | OA | 119 | 3 | 2.52 | — | — | — |
| | HC | 120 | 2 | 1.67 | — | — | — |
| Hangzhou cohort (validation | RA | 155 | 115 | 74.19 | 83.35 | 71.88 | 84.73 |
| cohort 2) | SLE | 80 | 13 | 16.25 | — | — | — |
| | SS | 55 | 23 | 41.82 | — | — | — |
| | OA | 32 | 7 | 21.88 | — | — | — |
| | HC | 100 | 2 | 2.00 | — | — | — |
| Pooled three cohorts | RA | 896 | 595 | 66.41 | 91.45 | 80.19 | 83.94 |
| | SLE | 478 | 63 | 13.18 | — | — | — |
| | SS | 319 | 60 | 18.81 | — | — | — |
| | OA | 223 | 15 | 6.73 | — | — | — |
| | HC | 700 | 9 | 1.29 | — | — | — |

The cut-off value of sSR-A was established 3 standard deviations above the mean value of healthy controls. The sensitivity and specificity of sSR-A for identification of RA were 61.36% and 94.38% in Beijing cohort, 73.24% and 90.51% in Inner Mongolia cohort, 74.19% and 83.15% in Hangzhou cohort, and 66.41% and 91.45% in the pooled three cohorts. RA means rheumatoid arthritis, SLE means systemic lupus erythematosus, SS means Sjogren's syndrome, OA means osteoarthritis, HC means healthy control, N means number of total patients, n means the number sSR-A positive patients, PPV means positive predictive value, NPV means negative predictive value.

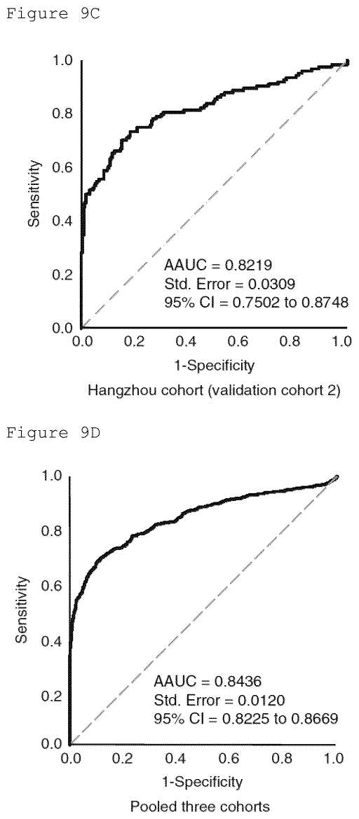

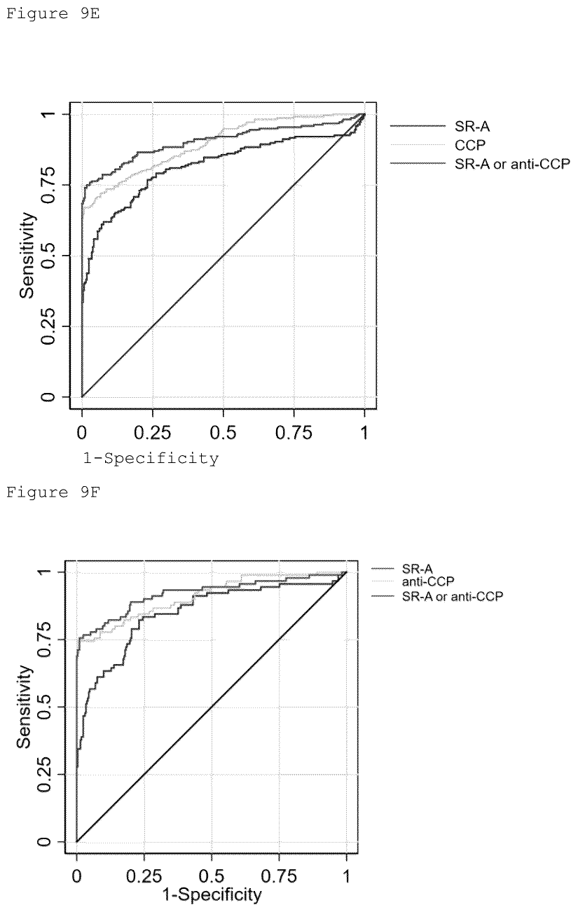

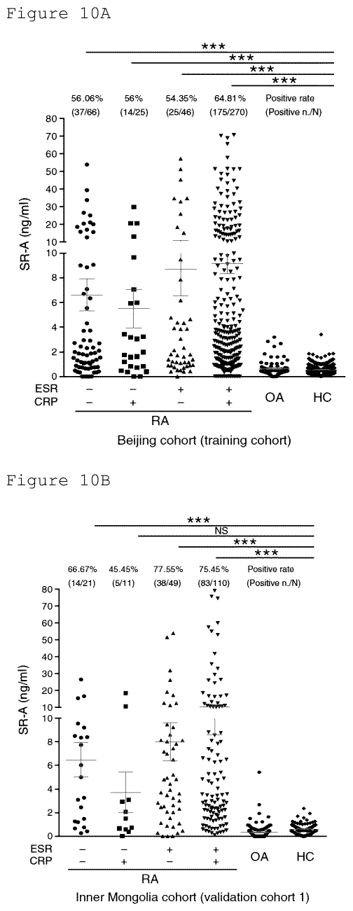

The performance of sSR-A was then compared with ESR and CRP, the two indexes listed in ACR/EULAR 2010 classification criteria. RA patients from the training and validation cohorts as well as the pooled cohort were divided into the following four groups, and the levels of sSR-A as well as the positive rates were further analyzed: RA patients with normal ESR and normal CRP, RA patients with normal ESR and increased CRP, RA patients with increased ESR and normal CRP, RA patients with increased ESR and increased CRP. The results showed that sSR-A demonstrated elevated levels with high prevalence in all these four groups. Even in RA patients with normal ESR and normal CRP, the positive rate of sSR-A still reached 57.58% (57/99) in the pooled three cohorts (see, FIGS. 10A, 10B, 10C, and 10D). All these results indicate that sSR-A provides a complementary value to ESR and CRP.

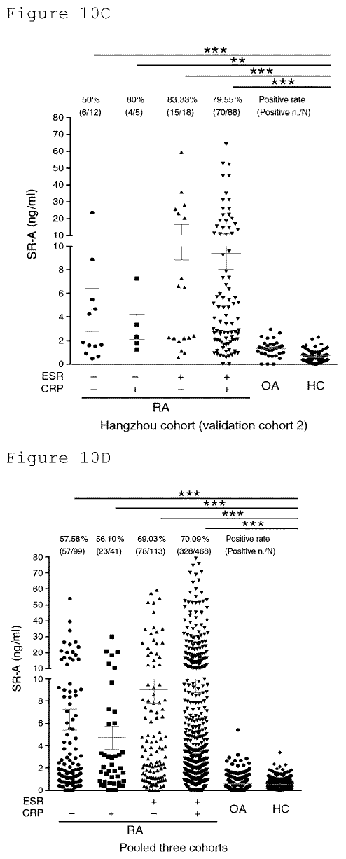

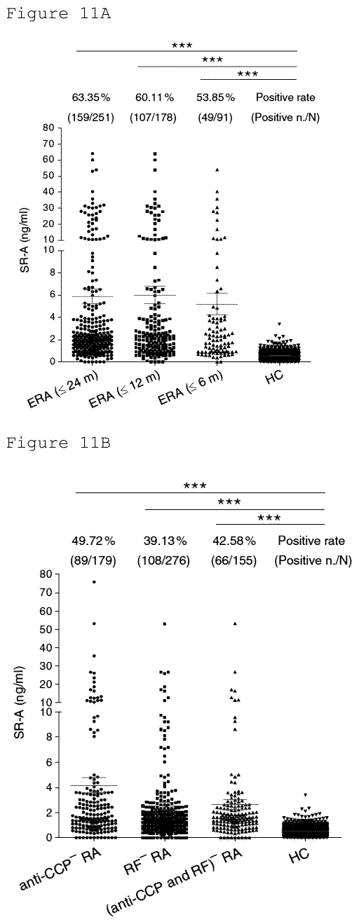

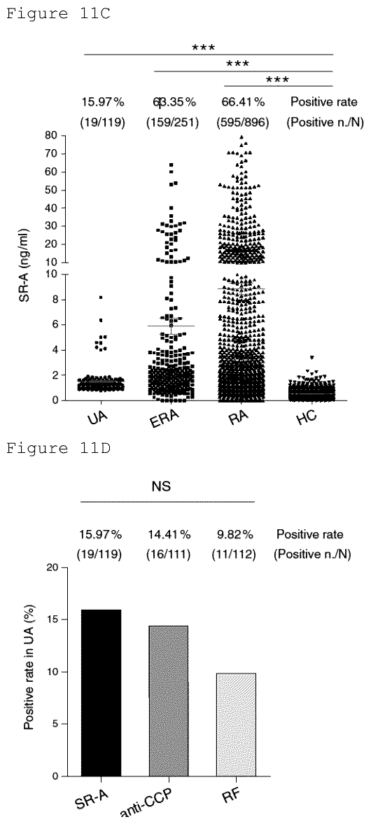

Early diagnosis and timely treatment of RA are critical for reducing co-morbidity and mortality. The diagnostic value of sSR-A in early-stage RA (ERA) was examined. In the three training and validation cohorts, there were 167 ERA patients in total with disease duration <24 months. To accurately elucidate the prevalence of sSR-A, an additional 84 ERA patient serum samples were collected from the Beijing cohort. In these 251 early RA patients, sSR-A showed substantial diagnostic value. In early RA patients with disease duration <12 and <24 months, the positive rates of sSR-A were 60.11% (107/178) and 63.35% (159/251), respectively. In early RA patients with disease duration <6 months, sSR-A also showed a 53.85% (49/91) prevalence (see, FIG. 11A).

Since anti-CCP and RF are routinely used for RA diagnosis, the complementary diagnostic value of sSR-A in patients lacking these two disease-specific antibodies were assessed. Serum samples from 179 anti-CCP-negative RA patients, 276 RF-negative RA patients, and 155 (anti-CCP and RF)-double negative RA patients (including samples from the three training and validation cohorts) were subjected to the detection of sSR-A levels and prevalence. The positive rates of sSR-A in anti-CCP-negative and RF-negative RA patients were 49.72% (89/179) and 39.13% (108/276), respectively. More importantly, in (anti-CCP and RF)-double negative RA patients, sSR-A also demonstrated a 42.58% (66/155) prevalence (see, FIG. 11B). These results support the use of sSR-A to facilitate the diagnosis in anti-CCP and/or RF-negative RA patients.

A study was also conducted to evaluate the potential of SR-A in combination with anti-CCP for the diagnosis of RA. 215 RA patients, 45 SLE patients, 30 SS patients, 37 OA patients, and 179 healthy volunteers with both SR-A and anti-CCP data were used for analysis. Combining SR-A with anti-CCP can improve the sensitivity of clinical diagnosis of RA. The sensitivity and specificity were 59.53% and 93.45% for SR-A, 66.97% and 98.28% for anti-CCP, and 79.07% and 91.72% for SR-A and anti-CCP combined in diagnosis of RA (Table 2). Similar results were observed in early RA (EPA) patients. The sensitivity and specificity were 54.26% and 93.45% for SR-A, 73.40% and 98.29% for anti-CCP, and 78.72% and 91.72% for the SR-A and anti-CCP combined in diagnosis of ERA (Table 3).

TABLE 2
Sensitivity and specificity of the combination
of SR-A and anti-CCP for RA diagnosis

| | Sensitivity | Specificity | PPV | NPV |
|---|---|---|---|---|
| | (%) | (%) | (%) | (%) |
| SR-A | 59.53 | 93.45 | 87.07 | 75.70 |
| Anti-CCP | 66.97 | 98.28 | 96.64 | 80.06 |
| RF | 64.65 | 87.93 | 79.89 | 77.04 |
| SR-A or Anti-CCP | 79.07 | 91.72 | 87.63 | 85.53 |
| SR-A and Anti-CCP | 47.44 | 100 | 100 | 71.96 |
| SR-A or RF | 67.91 | 83.10 | 74.87 | 77.74 |
| SR-A and RF | 50.70 | 97.59 | 93.97 | 72.75 |
| RF or Anti-CCP | 75.35 | 85.17 | 79.02 | 82.33 |
| RF and Anti-CCP | 56.28 | 98.97 | 97.58 | 75.33 |
| SR-A or Anti-CCP or RF | 80.93 | 82.76 | 77.68 | 85.41 |
| SR-A and Anti-CCP and RF | 44.19 | 100 | 100 | 70.73 |

TABLE 3
Sensitivity and specificity of the combination
of SR-A and anti-CCP for ERA diagnosis

| | Sensitivity | Specificity | PPV | NPV |
|---|---|---|---|---|
| | (%) | (%) | (%) | (%) |
| SR-A | 54.26 | 93.45 | 72.86 | 86.31 |
| Anti-CCP | 73.40 | 98.28 | 93.24 | 91.94 |
| RF | 57.45 | 87.93 | 60.67 | 86.44 |
| SR-A or Anti-CCP | 78.72 | 91.72 | 75.51 | 93.01 |
| SR-A and Anti-CCP | 48.94 | 100 | 100 | 85.80 |
| SR-A or RF | 67.02 | 83.10 | 56.25 | 88.60 |
| SR-A and RF | 44.68 | 97.59 | 85.71 | 84.48 |
| RF or Anti-CCP | 76.60 | 85.17 | 62.61 | 91.82 |
| RF and Anti-CCP | 54.26 | 98.97 | 94.44 | 86.97 |
| SR-A or Anti-CCP or RF | 78.72 | 82.76 | 56.68 | 92.31 |
| SR-A and Anti-CCP and RF | 41.49 | 100 | 100 | 84.06 |

The value of sSR-A as a predictor was also examined. Serum samples from 119 undifferentiated arthritis (UA) patients were further collected for sSR-A detection. Although lower than those in ERA and RA patients, the levels of sSR-A were moderately increased in UA patients as compared with healthy controls (see FIG. 11C). As shown at FIG. 11D, the prevalence of sSR-A in UA patients was 15.97% (19/119), comparable with anti-CCP (14.41%, 16/111) and RF (9.82%, 11/112). Moreover, those UA patients with high levels of sSR-A tended to display increased ESR or CRP, and positive RF. Comparing UA, ERA, and RA patients showed that the levels and/or positive rates of sSR-A were increased during disease progression (see, FIG. 11C). Those findings indicate the potential value of sSR-A as a predictor of early RA.

Taken together, all these results suggest that sSR-A represents a biomarker with the potential for an earlier and more accurate diagnosis of RA.

Correlation of sSR-A with RA Patient Manifestations.

Correlation analysis showed that sSR-A was associated with many clinical and immunological features of RA patients (see, Table 4). Especially, the levels of sSR-A positively correlated with serum RF and IgM in both the training and validation cohorts. In the Beijing cohort, the levels of sSR-A also correlated with glucose-6-phosphate isomerase (GPI). No obvious association was found between sSR-A and RA patient ages, CRP, and IgG.

TABLE 4
Association between sSR-A and RA patient clinical/immunological features

| | Beijing cohort | | Inner Mongolia cohort | | Hangzhou cohort | |
|---|---|---|---|---|---|---|
| Characteristics | r | p | r | p | r | p |
| Ages | 0.011 | 0.823 | −0.028 | 0.700 | 0.132 | 0.145 |
| Disease duration | 0.112 | 0.019* | 0.078 | 0.285 | 0.054 | 0.555 |
| ESR | 0.170 | 4.590E−04*** | 0.138 | 0.058 | 0.240 | 0.007** |
| CRP | 0.099 | 0.043* | 0.026 | 0.724 | 0.147 | 0.104 |
| DAS28 | 0.117 | 0.017* | 0.091 | 0.221 | 0.137 | 0.130 |
| IgA | 0.117 | 0.017* | 0.188 | 0.010* | 0.190 | 0.036* |
| IgM | 0.311 | 8.327E−11*** | 0.221 | 0.002** | 0.158 | 0.083 |
| IgG | 0.020 | 0.689 | 0.094 | 0.201 | 0.143 | 0.116 |
| RF | 0.622 | 7.599E−46*** | 0.506 | 1.215E−13*** | 0.584 | 1.288E−12*** |
| Anti-CCP | 0.293 | 3.313E−09*** | 0.083 | 0.261 | 0.145 | 0.115 |
| Albumin | −0.165 | 7.330E−04*** | 0.282 | 0.090 | — | — |
| WBC | 0.102 | 0.035* | 0.018 | 0.912 | — | — |
| GPI | 0.505 | 1.093E−26*** | — | — | — | — |

The important items with significant associations are indicated by bold. *P<0.05, **p<0.01, ***p<0.001, with exact p values shown in the table (two-tailed Spearman's rank correlation test). ESR means erythrocyte sedimentation rate, CRP means C-reactive protein, DAS28 means disease activity score 28, Ig means immunoglobulin, RF means rheumatoid factor, Anti-CCP means anti-cyclic citrullinated peptide antibody, WBC means white blood cell, GPI means gloucose-6-phosphate isomerase, AKA means antikeratin antibodies, APF means antiperinuclear factor antibodies.

The RA patients were then divided into sSR-A-positive and sSRA-negative groups by the cut-off value. Detailed analyses showed that the levels of rheumatoid factors (RF), immunoglobulin M (IgM), and glucose-6-phosphate isomerase (GPI) were significantly higher in the sSR-A-positive group than in the sSR-A-negative group, consistent with the associations as described above (see, Table 1).

There was also a modest correlation between sSR-A levels and RA patient radiographic damage as assessed by the Sharp/van der Heijde score (SHS). Moreover, sSR-A positive RA patients showed relatively higher SHS than sSR-A negative RA patients.

To further confirm these findings, the levels of sSR-A in both non-responders (DAS28>5.1) and responders (DAS28<2.6) of RA patients after therapy were assessed, and their clinical correlations were analyzed, respectively. The levels of sSR-A were significantly decreased in the responders but not in the non-responders of RA patients after therapy. Moreover, these correlations as described above were more evident in the non-responders, yet could not be seen in the responders.

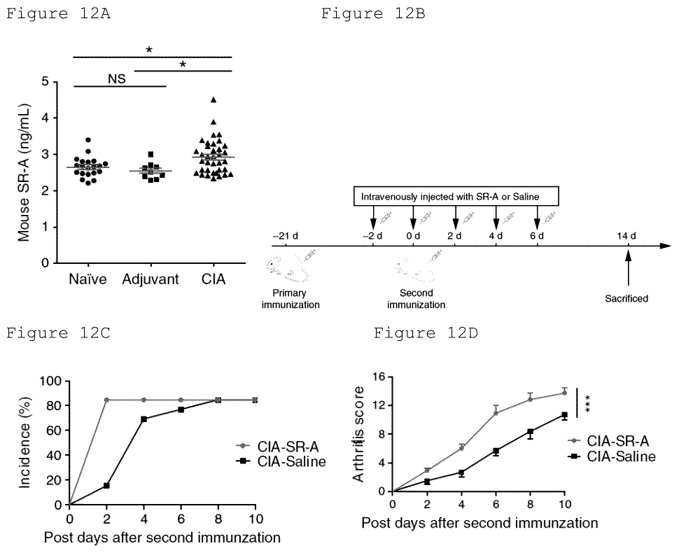

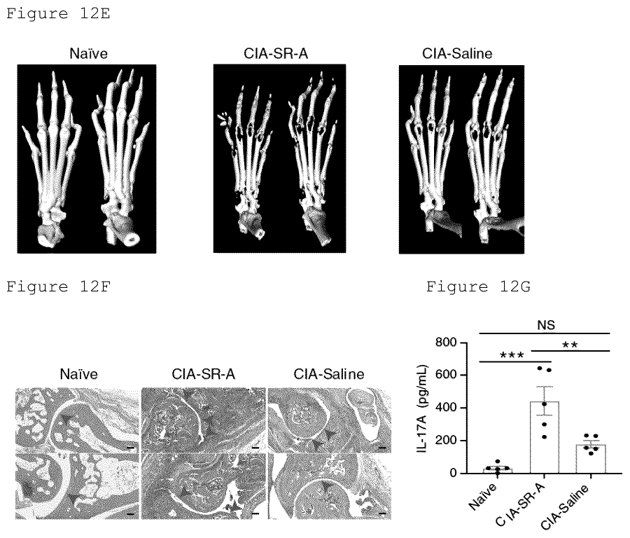

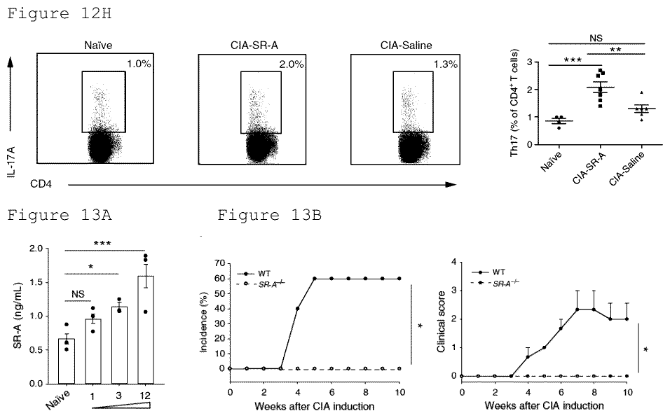

Elevation of SR-A exacerbates autoimmune arthritis in mice. The role of sSR-A in disease pathogenesis using mouse arthritis models was investigated. Upon collagen-induced arthritis (CIA) in DBA/1 mice, there was a significant elevation of sSR-A in the serum as compared with that in naïve mice or adjuvant immunized mice (see, FIG. 12A). To further examine the activity of sSRA, intravenous injections of recombinant SR-A protein (i.e., extracellular domain of SR-A) into DBA/1 mice (2 μg/mouse) every 2 days starting from 2 days before boosting immunization for a total of five doses (see, FIG. 12B). Surprisingly, the mice receiving recombinant SR-A protein showed earlier disease onset as well as significantly higher clinical scores as compared with those control mice receiving saline (see, FIGS. 12C and 12D). This disease-exacerbating effect was abolished when the recombinant SR-A protein was boiled prior to administration, excluding the possibility of endotoxin contamination. Supplementing SR-A protein in CIA mice also resulted in more severe bone destruction as assessed by micro-CT (see, FIG. 12E), and promoted the inflammation and pannus infiltrates in the joints (see, FIG. 6F). Evaluation of immune cells in lymphoid organs showed that injection of SR-A protein caused elevation of IL-17A in the serum associated with increased frequency of IL-17A-producing CD4+ T cells (see, FIGS. 12G and 12H), implicating a linkage of the sSR-A activity with a pathogenic Th17 response in arthritis. The activity of sSR-A in accelerating arthritis progression and Th17 inflammatory responses was also independently confirmed using a higher dose of SR-A protein (30 μg/mouse).

### SR-A Ablation Abrogates Autoimmune Arthritis in Mice.

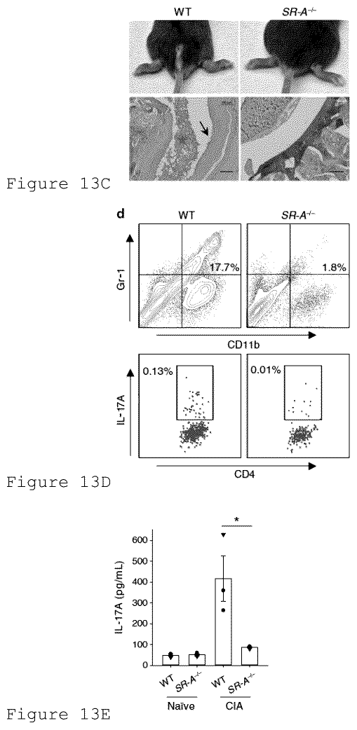

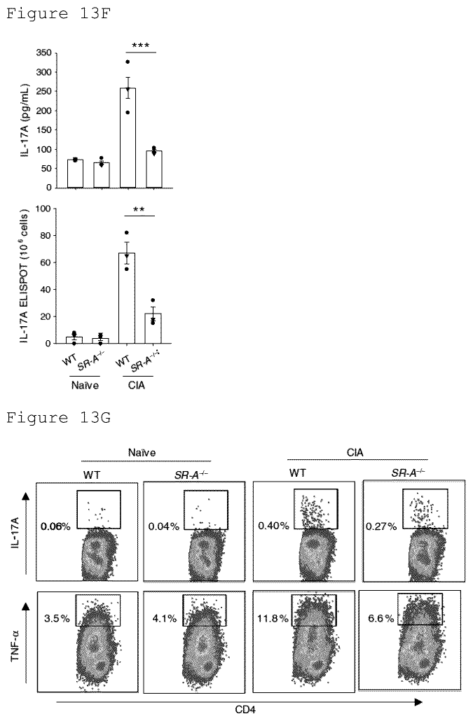

Using SR-A-deficient mice on a C57BL/6 background, additional studies to validate the role of SR-A in arthritis development were performed. Consistent with the results from DBA/1 mice, there was an evident increase of serum sSR-A during disease progression, which positively correlated with clinical scores of arthritis (see, FIG. 13A). Ablation of SR-A rendered mice fully protected from CIA (see, FIG. 13B). Absence of SR-A also abolished cartilage erosion and inflammatory exudation in the articular cavity (see, FIG. 13C), which was associated with decreased infiltration of inflammatory myeloid cells and Th17 cells in the joint (see, FIG. 13D), further implicating SR-A as a factor that can drive inflammatory and pathogenic processes in arthritis. Additionally, absence of SR-A impaired the Th17 response in arthritic mice, indicated by reduced IL-17A levels in the blood (see, FIG. 13E), diminished production of IL-17A by splenocytes upon collagen stimulation (see, FIG. 13F), and decreased frequency of collagen-reactive Th17 cells in the spleens (see, FIG. 13G). Intracellular cytokine staining of splenocytes showed that, in addition to Th17 cells, lack of SR-A also dramatically reduced the frequency of Th1 cells, i.e., TNF-α-producing CD4+ T cells (see, FIG. 13G), underscoring an important role of SR-A in arthritis-associated inflammation.

To exclude the possibility that SR-A deficiency might affect the intrinsic immunogenicity of antigen-presenting cells during collagen immunization, the DC activity and collagen specific immune responses were investigated. No difference was seen between WT and SR-A−/− mice in the frequency and phenotype of DCs, collagen-reactive IFN-γ production, or a collagen-specific humoral response, suggesting that lack of SR-A in the model of CIA does not alter antigen-presenting function or antigen trafficking.

The relationship between IL-17A and sSR-A was investigated by neutralization of IL-17A in WT CIA mice. SR-A−/− CIA mice were also treated with IL-17A-neutralizing antibody as controls. It was shown that IL-17A neutralization substantially ameliorated the disease severity in WT mice. However, neutralization of IL-17A failed to reduce the levels of serum sSR-A. Additionally, incubation of bone marrow-derived macrophages (BMMϕ) and bone marrow-derived dendritic cells (BMDCs), both of which highly express SR-A on cell surface, with inflammatory cytokines (i.e., IL-1β, IL-6, IL-17A, and TNF-α) failed to induce detectable secretion of sSR-A into the culture medium, suggesting that elevation of sSR-A may not be directly triggered by inflammatory cytokines.

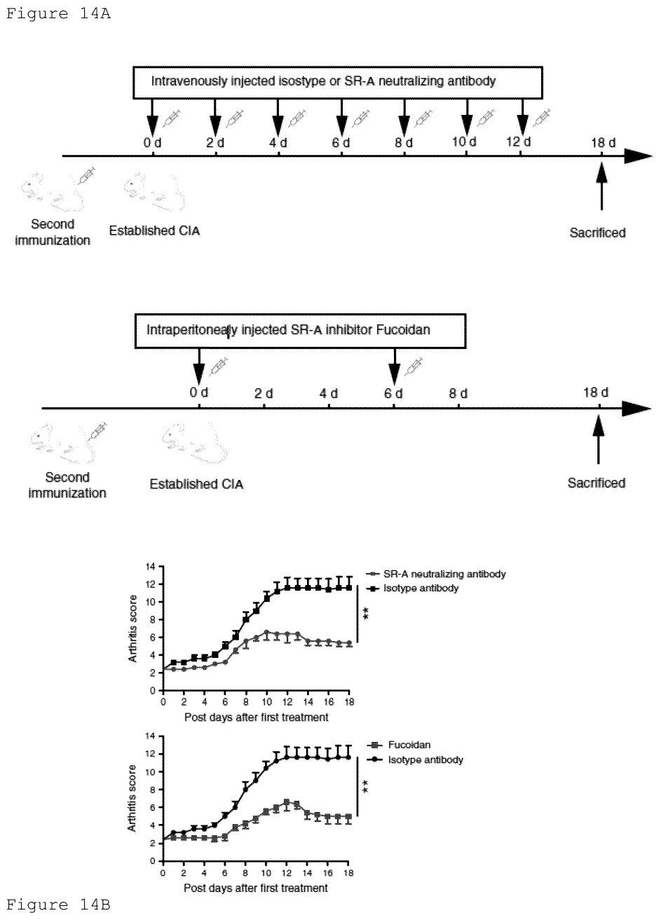

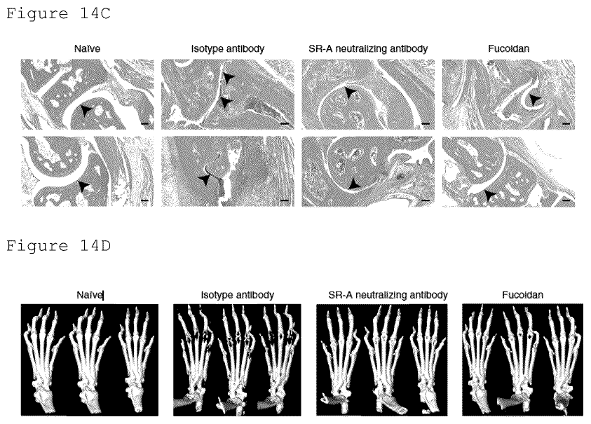

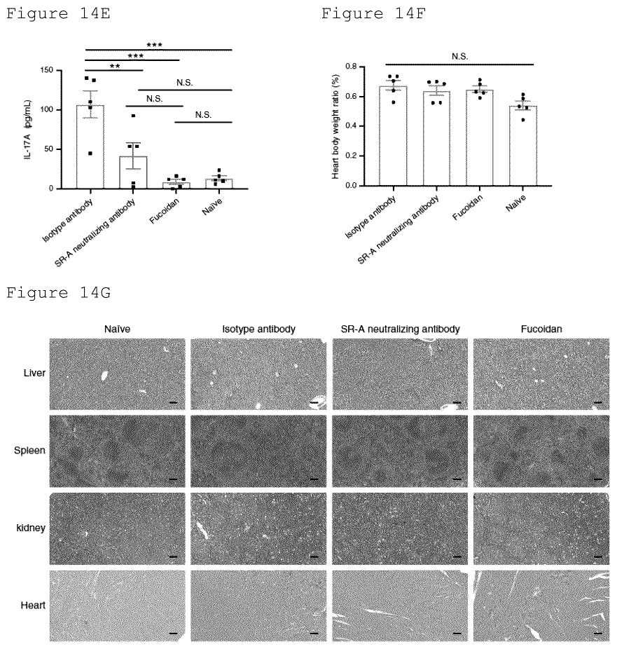

Inhibition of SR-A ameliorates the severity of arthritis. Given the role of SR-A in promoting the disease progression and/or severity, the SR-A serves as a therapeutic target for RA. The blockade of SR-A in CIA mice with either SR-A-neutralizing antibody or SR-A inhibitor Fucoidan as shown in FIG. 14A significantly reduced the severity of CIA (see, FIG. 14B). This SR-A inhibition also compromised the joint inflammation and bone destruction, as shown by the H&E staining and micro-CT imaging (see, FIGS. 14C and 14D). Remission of clinical symptoms by SR-A inhibition was also associated with significantly decreased IL-17A in the serum (see, FIG. 14E). Function and pathology evaluation of the heart, liver, spleen, and kidney showed that no significant side effects or tissue damage were observed after treatment with SR-A-neutralizing antibody or Fucoidan (see, FIGS. 14F and 14G).

### Patients and Samples

Serum samples were obtained from 1117 RA patients (including 251 early RA, 179 anti-CCP-negative RA, 276 RF-negative RA, and 155 anti-CCP & RF-negative RA), 478 SLE patients, 319 SS patients, 223 OA patients, 119 UA patients, 39 AS patients, 39 Gout patients, 39 PsA patients, 39 AAV patients, 30 AOSD patients, 24 PMR patients, 25 AIH patients, 71 non-autoimmune inflammatory disease patients, and 700 healthy volunteers. All patients fulfilled 2010 American College of Rheumatology (ACR) and European League Against Rheumatism (EULAR) criteria for RA, all SLE (SLICC Revision of ACR 2009), SS (ACR 2012), OA (ACR 1995), AS (Modified New York criteria 1984), Gout (ACR/EULAR 2015), PsA (CASPAR 2006), AAV (CHCC 2012), AOSD (Yamaguchi 1992), PMR (EULAR/ACR 2015), UA (EULAR 2007), AIH (IAIHG 1999), and non-autoimmune inflammatory disease patients fulfilled their classification criteria, respectively. The detailed characteristics of patients with RA are provided in Tables 5.

Radiological damage of patients with RA was evaluated using Sharp-van der Heijde (SHS) total score.

TABLE 5
Clinical and demographic characteristics of RA patients in the diagnostic study.

| | Beijing cohort | | Inner Mongolia | | Hangzhou | |
|---|---|---|---|---|---|---|
| Characteristics | (n = 528) | | cohort (n = 213) | | cohort (n = 155) | |
| Age, median (range), yrs | 58 | (18-89) | 59 | (22-83) | 60 | (34-82) |
| Sex, female/male | 399/129 | | 157/56 | | 121/34 | |
| Disease duration, | 10 | (0.1-60) | 9 | (0.04-40) | 7 | (0.1-50) |
| median (mange), yrs | | | | | | |
| ESR, median (range), mm/h | 43 | (2-140) | 53 | (3-125) | 57 | (4-140) |
| CRP, median (range), mg/L | 20.2 | (0.21-741) | 16.8 | (0.6-181) | 22.6 | (0.23-190.7) |
| Tender joint count of 28 | 5 | (0-28) | 11 | (0-28) | 7.5 | (0-28) |
| joints, median (range) | | | | | | |
| Swollen joint count of 28 | 4 | (0-28) | 2 | (0-28) | 2 | (0-28) |
| joints, median (range) | | | | | | |
| DAS28, median (range) | 4.9 | (0.99-8.40) | 5.55 | (0.99-8.33) | 5.25 | (2.26-8.67) |
| Medication, no. (%) | | | | | | |
| Steroids | 193 | (36.55) | 128 | (60.09) | 99 | (63.87) |
| NSAIDs | 274 | (51.89) | 39 | (18.31) | 55 | (35.48) |
| Methotrexate | 216 | (40.91) | 103 | (48.36) | 64 | (41.29) |
| Other DMARDs | 498 | (94.32) | 165 | (77.46) | 140 | (90.32) |
| Biologics | 56 | (10.61) | 15 | (7.04) | 7 | (4.52) |
| No treatment | 4 | (0.76) | 0 | (0) | 1 | (0.65) |

896 RA patients were recruited overall (528 in Beijing cohort, 213 in Inner Mongolia cohort, and 155 in Hangzhou cohort) ESR means erythrocyte sedimentation rate, CRP means C-reactive protein, DAS28 means disease activity score 28

### Mice

Six to eight-week-old male DBA/1 mice were purchased from Huafukang Bioscience Company (Beijing, China). All mice were housed in a specific pathogen-free environment under controlled conditions (22° C. ambient temperature, 40% humidity). All animal procedures complied with relevant ethical regulations for animal research and were approved by the Institutional Animal Care and Use Committee (IACUC) of Peking University People's Hospital.

### Enzyme-Linked Immunosorbent Assay (ELISA) Analysis

The serum levels of sSR-A in patients with RA and healthy individuals were detected by ELISA, using a commercially available human SR-A ELISA kit (Sino Biological Inc., Beijing, China). The levels of IL-6 in CIA mouse serum and the cell culture supernatant of patients with RA were detected by ELISA kit from Neobioscience (Beijing, China), while the levels of C-terminal telopeptide in CIA mouse serum were detected by ELISA kit from Chondrex (Redmond, WA). The results were obtained on a Synergy™ 4 Multi-Mode Microplate Reader with software GEN5CH 2.0 (BioTek, Winooski, VT).

### Collagen-Induced Arthritis Induction and Treatment

Collagen-induced arthritis (CIA) models were established in DBA/1 mice by immunization on Day 1 with 200 μg bovine type II collagen (Chondrex Inc., Redmond, WA) emulsified in complete Freund adjuvant (CFA), and on Day 21 with 100 μg bovine type II collagen emulsified in incomplete Freund adjuvant (IFA) as a booster. The severity of arthritis was scored based on the level of inflammation as described previously.

After the arthritis initiation, the mice with similar scores of 2-3 were randomly divided into two groups. As reported in previous studies concerning blocking antibodies, the experimental group mice were intravenously administrated with anti-mouse SR-A neutralizing antibodies (AF1797, R&D Systems, Minneapolis, MN; 2 μg/mice in 200 μl PBS), while the control group mice were intravenously administrated with isotype control IgG every other day for a total of five times. The mice were sacrificed on Day 12 after the first treatment for further analysis.

### Osteoclasts (OCs) Differentiation

For human OC differentiation in vitro, freshly isolated peripheral blood (PB) mononuclear cells (PBMCs) from patients with RA or healthy individuals were suspended in complete α-MEM containing 10% FBS and planted in a 96-well plate at a density of 1×10³ cells/well. After culturing for 4 h, the non-adherent cells were removed, remaining the adherent OC precursors. Then the cells were co-cultured with 2 μg/ml SR-A neutralizing antibody or isotype antibody along with RANKL (100 ng/ml) and M-CSF (50 ng/ml). Sometimes, the cells were co-cultured with RA serum pre-incubated with SR-A neutralizing antibody or isotype antibody in the presence of RANKL and M-CSF. The medium was changed every 3 days, and on Day 14, OC formation was measured through TRAP staining with leukocyte acid phosphatase kit (obtainable through Sigma-Aldrich, St. Louis, MO). TRAP-positive multinucleated cells (MNCs) containing three or more nuclei in the entire single well of 96-well plate were observed and counted under an inverted fluorescence microscope (Olympus IX71-141, Tokyo, Japan).

For mouse OC differentiation in vitro, primary bone marrow cells (BMMs) from CIA or na.ve mice were cultured in a 96-well plate at a density of 1×10³ cells/well for 3 days in complete α-MEM with 10% FBS and 25 ng/ml M-CSF. Then non-adherent cells were discarded and the adherent cells were further cultured in the presence of 50 ng/ml RANKL, 25 ng/ml M-CSF, 2 μg/ml SR-A neutralizing antibody or isotype antibody. Six days later, tartrate-resistant acid phosphatase (TRAP) staining was performed for OC detection.

For mouse OC differentiation ex vivo, BMMs from CIA mice or naïve mice treated with SR-A neutralizing antibody or isotype antibody as described above were collected for OC formation accordingly.

### Bone Resorption Assay

Peripheral blood mononuclear cells (1×10³ cells/well) from patients with RA were performed for OC differentiation as described above in 96-well plates coated with bovine cortical bone slices (6 mm in diameter and approx. 200 μd thick, IDS, Boldon, UK). On Day 14, the bone slices containing the OCs were collected and washed with distilled water. Then, the resorption pits were visualized by staining with 1% toluidine blue and detected under a polarized light microscope (Olympus BX51-P, Tokyo, Japan). The results were expressed as the area of resorption lacunae in the total plate area.

### Mouse Paw Histophathology and Micro-CT Analysis

Mice were sacrificed and the hind paws were fixed in 4% buffered formaldehyde. The tissues were collected, fixed, paraffin-embedded, sectioned, stained with TRAP, and analyzed with NDP.view2 (Hamamatsu Photonics K.K., Japan).

Micro-CT images of the mouse paws were acquired on the Tri-Modality FLEX Triumph™ Pre-Clinical Imaging System (Gamma Medica-Ideas, Northridge, CA). CT image set acquisitions lasted 10 min and utilized beam parameters of 130 μA and 80 kVP. Analyze 10.0 (AnalyzeDirect, Overland Park, KS) was used to perform the image analysis.

Reverse Transcription-Polymerase Chain Reaction (RT-PCR) and Quantitative Polymerase Chain Reaction (qPCR)

RNA extraction, reverse transcription, and qPCR analyses were performed as described previously. Briefly, after the formation of OCs as described above, total RNA was extracted by RNAsimple Total RNA kit (Tiangen, Beijing, China), and reverse transcription was performed with the reverse transcriptase kit (Thermofisher Scientific, Waltham, MA) according to the manufacturer's instructions. Then, qPCR was performed to analyze the OC-specific gene expression using Power SYBR Green PCR Master Mix (Applied Biosystems, Foster City, CA). The primers used were as follows:

Human GAPDH sense primer:
5′-AAGGTGAAGGTCGGAGTCAA-3′,
antisense primer:
5′-AATGAAGGGGTCATTGATGG-3′;
Human TRAP sense primer:
5′-GATCCTGGGTGCAGACTTCA-3′,
antisense primer:
5′-GCGCTTGGAGATCTTAGAGT-3′;
Human CTSK sense primer:
5′-ACCGGGGTATTGACTCTGAA-3′,
antisense primer:
5′-GAGGTCAGGCTTGCATCAAT-3′;
Human MMP-9 sense primer:
5′-CGCTACCACCTCGAACTTTG-3′,
antisense primer:
5′-GCCATTCACGTCGTCCTTAT-3′.
Mice GAPDH sense primer:
5′-GGTGAAGGTCGGTGTGAACG-3′,
antisense primer:
5′-CTCGCTCCTGGAAGATGGTG-3′;
Mice TRAP sense primer:
5′-CGACCATTGTTAGCCACATACG-3′,
antisense primer:
5′-TCGTCCTGAAGATACTGCAGGTT-3′;
Mice CTSK sense primer:
5′-GCTTGGCATCTTTCCAGTTTTAC-3′,
antisense primer:
5′-TATCCAGTGCTTGCTTCCCTTCT-3′;
Mice MMP-9 sense primer:
5′-CGTGTCTGGAGATTCGACTTGA-3′,
antisense primer:
5′-TTGGAAACTCACACGCCAGA-3′.

### Statistical Analysis

GraphPad Prism 8 (GraphPad Software Inc., San Diego, CA) was used for statistical analysis. Differences between various groups were evaluated by the Spearman's rank correlation test, Mann-Whitney U test, Wilcoxon matched-paired signed-rank test, paired Student's t-test, and one-way ANOVA test. All data are expressed as mean±SEM.A confidence level above 95% (P<0.05) was considered statistically significant (*P<0.05, **P<0.01, ***P<0.001, ns, not significant).

### Results

Elevated Serum sSR-A Correlates with Bone Destruction of the Patient with RA

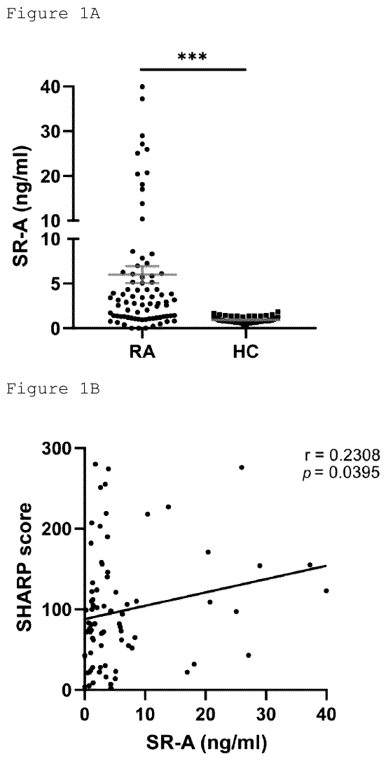

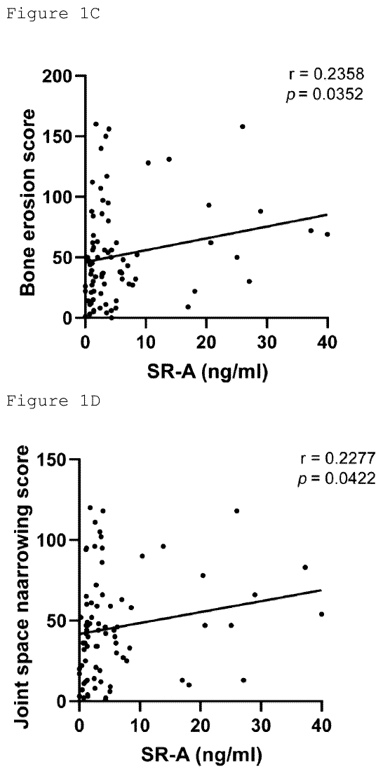

The diagnostic value of sSR-A and its pathogenic activity in RA and the correlation between sSR-A and bone destruction in patients with RA have been confirmed. Serum samples from patients with RA with radiographic images (n=80) and healthy controls (HC), (n=60), were collected for detection of the levels of sSR-A. The result showed that sSR-A in patients with RA was significantly higher than that in HC (median 2.906 ng/ml, mean 6.006 ng/ml, SD 8.441 ng/ml, ***P<0.001, see, FIG. 1A). Sharp/van der Heijde score (SHS) was then performed and simple linear regression showed that the level of sSR-A positively correlated with the SHARP score (see, FIG. 1B). Further analysis showed that the serum sSR-A level in patients with RA correlated positively with both erosion score and JSN score (see, FIGS. 1C and 1D).

SR-A Neutralizing Antibody Inhibits OC Differentiation and Bone Destruction in Patients with RA

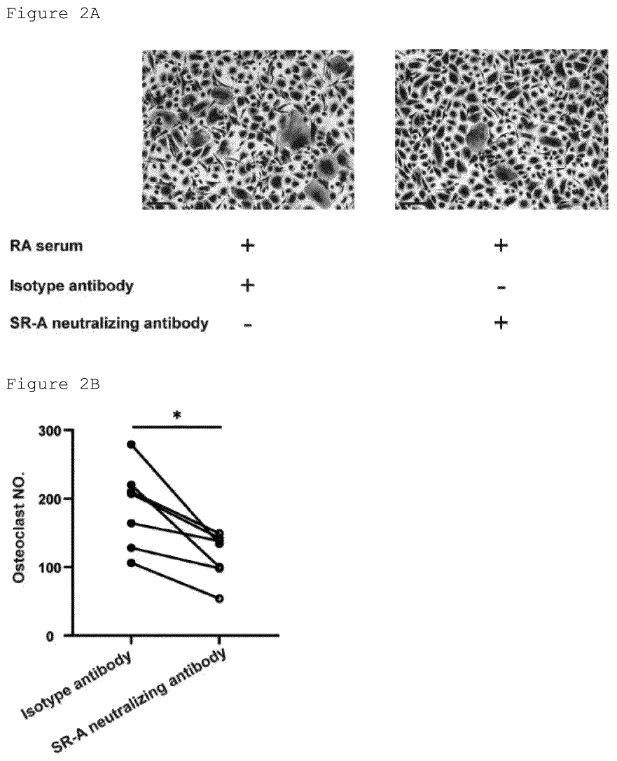

The potential involvement of sSR-A in RA bone destruction was uncovered. As shown at FIGS. 2A and 2B, treatment of healthy individual monocytes with serum of the patient with RA fundamentally stimulated OC formation in vitro. However, the stimulatory effects of serum of the patients with RA were aborted when pre-incubated with SR-A neutralizing antibody, which indicated the role of sSR-A in osteoclastogenesis.

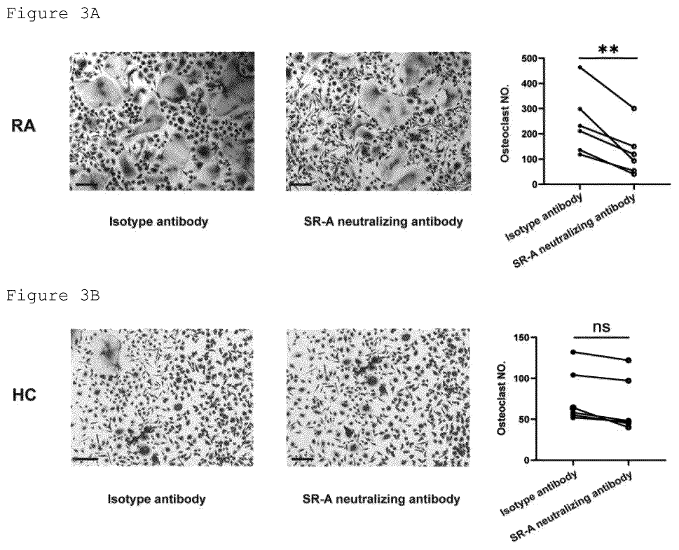

The effects of blockade of SR-A on osteoclastogenesis in patients with RA were examined. Monocytes from patients with RA and healthy individual peripheral blood were cultured with SR-A neutralizing antibody or isotype antibody for OC differentiation. Tartrate-resistant acid phosphatase (TRAP) staining showed that compared with isotype antibody, SR-A neutralizing antibody could substantially inhibit the OC formation and decrease the number of OCs in patients with RA (see, FIG. 3A). Nevertheless, only a minimal inhibitory effect of SR-A neutralizing antibody on OC differentiation was seen in healthy individuals (see, FIG. 3B), indicating the disease-specific pathologic role of SR-A. Bone resorption assay further showed that SR-A neutralizing antibody could functionally dampen the bone erosion and destruction in patients with RA (see, FIG. 3C). Moreover, the expression of TRAP, cathepsin K (CTSK), and matrix metalloproteinase-9 (MMP-9), marker genes for OCs, were significantly down-regulated after SR-A neutralizing antibody treatment in RA (see, FIG. 3D). The levels of IL-6 in the cell culture supernatants were also fundamentally decreased after the treatment (see, FIG. 3E).

Although SR-A neutralizing antibody blocks both soluble and membrane-bound SR-A, the results revealed that the expression of membrane-bound SR-A on monocytes of the patient with RA were significantly decreased as compared with healthy individuals, while the levels of SR-A mRNA as well as soluble SR-A in the serum were significantly elevated (data not shown). Therefore, the data indicates SR-A neutralizing antibodies inhibit osteoclastogenesis in patients with RA mainly through blocking soluble SR-A.

### SR-A Neutralizing Antibody Dampens OC Differentiation in Arthritis Mice In Vitro

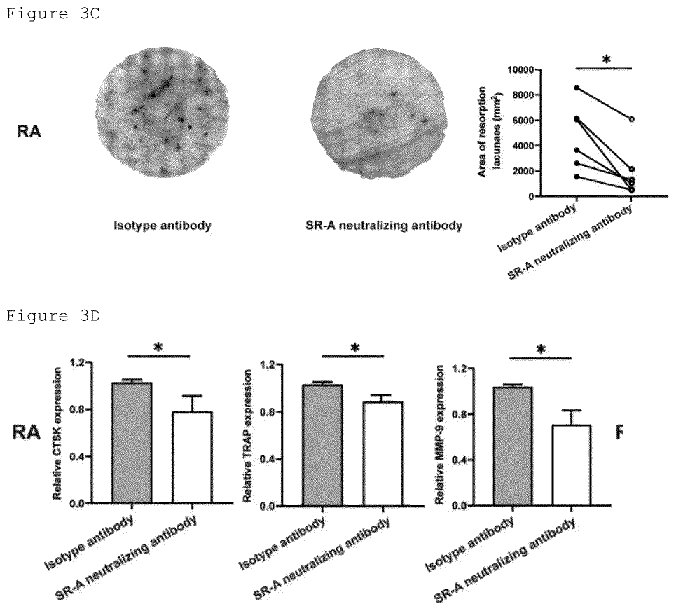

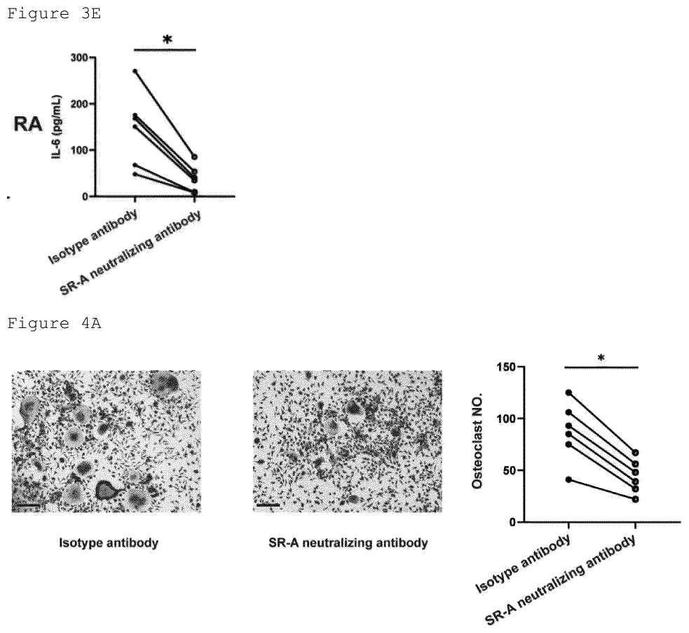

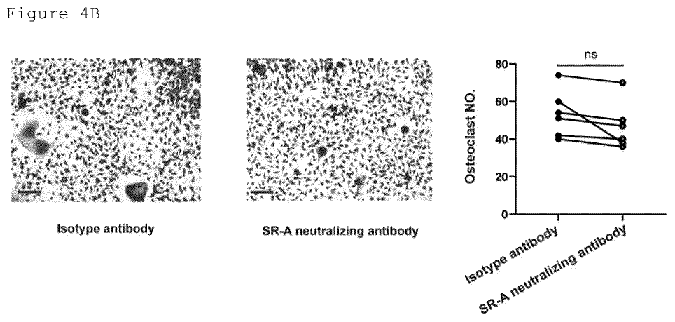

The effects of SR-A neutralizing antibody on OC differentiation in collagen-induced arthritis (CIA) mice in vitro were detected. Bone marrow-derived monocytes (BMMs) from CIA mice and naïve mice were cultured with SR-A neutralizing antibody or isotype antibody for 9 days in the presence of M-CSF and RANKL. As shown at FIG. 4A, TRAP staining revealed that SR-A neutralizing antibody could markedly alleviate the OC formation in CIA mice. However, only a faintest effect was detected in naïve mice (see, FIG. 4B), similar to that in healthy individuals. These results further demonstrated the function of SR-A neutralizing antibodies in compromising OC differentiation in RA.

### SR-A Neutralizing Antibody Ameliorates Osteoclastogenesis in Arthritis Mice In Vivo

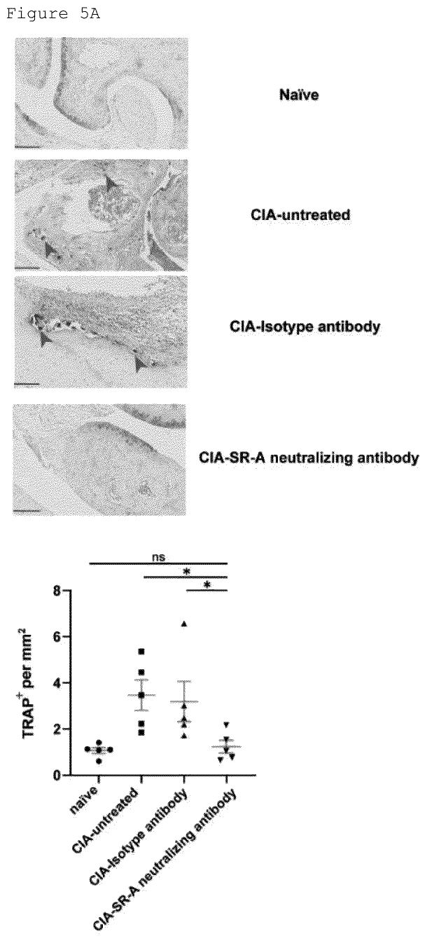

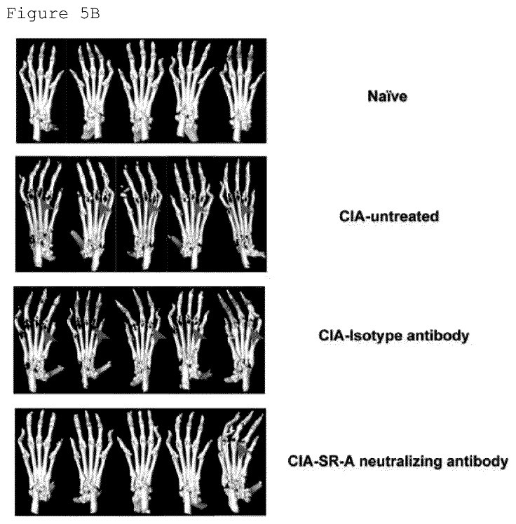

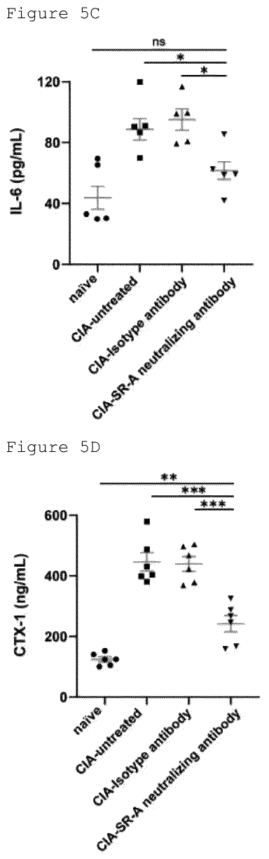

It was revealed that the blockade of SR-A by SR-A neutralizing antibodies could affect OC formation and bone destruction in CIA mice in vivo. Established CIA mice with similar scores of 2-3 was injected with SR-A neutralizing antibody or isotype antibody intravenously once every 2 days for five injections before sacrifice. As shown at FIG. 5A, TRAP staining revealed that SR-A neutralizing antibodies could decrease the number of OCs in CIA mice. Micro-CT imaging further demonstrated that compared with isotype antibody, SR-A neutralizing antibody also significantly alleviated bone destruction (see, FIG. 5B). The data also confirmed that the serum levels of IL-6 that promote osteoclastogenesis, as well as the serum levels of C-terminal telopeptide, a biomarker for bone remodeling, were significantly decreased in CIA mice receiving SR-A neutralizing antibody treatment (see, FIGS. 5C and 5D). All these results indicated the function of SR-A neutralizing antibody in ameliorating arthritis severity and osteoclastogenesis in vivo.

### SR-A Neutralizing Antibody Attenuates OC Differentiation in Arthritis Mice Ex Vivo

To confirm the direct effects of SR-A neutralizing antibody on osteoclastogenesis in CIA mice in vivo, BMMs from CIA mice receiving SR-A neutralizing antibody or isotype antibody treatment as described above were harvested for OC differentiation ex vivo. TRAP staining showed that the number of OCs in CIA mice treated with SR-A neutralizing antibody was significantly lower than that in CIA mice treated with isotype antibody (see, FIG. 6A). Moreover, the expression of OC-specific-marker genes, CTSK, TRAP, MMP-9, was also down-regulated after SR-A neutralizing antibody treatment (see, FIG. 6B).

Taken together, these results revealed the direct function of SR-A neutralizing antibodies in ameliorating arthritis severity and osteoclastogenesis in vivo.

### Discussion

This data has revealed the diagnostic value and pathogenic activity of SR-A in patients with RA and mice with experimental arthritis; and conveyed a positive correlation between sSR-A and bone destruction in patients with RA. Moreover, the data revealed the therapeutic potential of blocking SR-A by neutralizing antibodies in ameliorating OCs differentiation and bone destruction both in patients with RA and CIA mice.

It was determined that therapeutically effective amount of anti-SR-A neutralizing antibodies in patients or subjects experiencing RA should range from: (i) 0.1 mg/day to 10 mg/day; (ii) 0.1 mg/every other day to 10 mg/every other day; (iii) 0.1 mg/once a week to 10 mg/once a week; (iv) 0.1 mg/once a month to 10 mg/once a month; (v) combinations thereof. Notice that administering the therapeutically effective amount of anti-SR-A neutralizing antibodies is to an RA and/or EPA diagnosed subject. The subject could be administered therapeutically effective amount of anti-SR-A neutralizing antibodies (a) just after being diagnosed with RA and/or ERA, or (b) any time after being diagnosed with RA and/or ERA.

The above-identified equipment can be contained in a kit. The kit contains a vial made of plastic, metal, glass, or combinations thereof that contains a therapeutically effective amount of anti-SR-A neutralizing antibodies to be administered to a subject having a diagnosis of rheumatoid arthritis (PA) or early-stage rheumatoid arthritis (ERA).

The kit can also have a syringe. The syringe is a conventional syringe or equivalent thereof that is capable of withdrawing or obtaining a liquid biological sample from the subject. As previously expressed, the liquid biological sample is selected from the group consisting of serum, plasma, synovial fluid, and combinations thereof.

The kit's container can also include an immunoassay device, for example and not limited to an immunoassay plate. After the syringe collects the liquid biological sample then, for example, the syringe or equivalent thereof transfers the liquid biological sample into or on to the immunoassay plate. In other words, the immunoassay device receives the liquid biological sample. Once the immunoassay device (or plate) receives the liquid biological sample, the immunoassay device permits the liquid biological sample to be (a) incubated (if necessary), (b) mixed with known reagents and/or separated (if necessary) to interact with and/or isolate the soluble macrophage scavenger receptor A (sSR-A) and/or macrophage scavenger receptor A (SR-A). The liquid biological sample in the immunoassay device (plate) is then measured, as identified above, to quantify an amount of soluble macrophage scavenger receptor A (sSR-A) and/or macrophage scavenger receptor A (SR-A) level contained in the liquid biological sample. And when the measurement of sSR-A and/or SR-A from the liquid biological sample is 1.7024 ng/ml or greater, and with greater certainty when the measurement of sSR-A and/or SR-A from the liquid biological sample is 5 ng/ml or greater, then the subject is diagnosed with RA and/or ERA.

The immunoassay device, for example and not limited to an instant ELISA kit designed to measure sSR-A and/or SR-A, receives at least a portion of the liquid biological sample. The liquid biological sample is incubated, and then measured through conventional instrumentation—as described above—the results were obtained on a Synergy™ 4 Multi-Mode Microplate Reader with software GEN5CH 2.0 (BioTek, Winooski, VT). That readout measures the sSR-A and/or the SR-A content and/or levels from a liquid biological sample obtained from a patient or subject. As indicated above, the immunoassay device can be any conventional immunoassay format that permits a person to determine the amount of sSR-A and/or SR-A measurement in the liquid biological sample is 1.7024 ng/ml or greater, and with greater certainty when the measurement of sSR-A and/or SR-A from the liquid biological sample is 5 ng/ml (or 7 ng/ml with even more certainty) or greater. When a physician, technician, physician's assistant, nurse, or equivalent thereof can recognize the amount of sSR-A and/or SR-A measurement in the liquid biological sample is 1.7024 ng/ml or greater, and with greater certainty when the measurement of sSR-A and/or SR-A from the liquid biological sample is 5 ng/ml (or 7 ng/ml with even more certainty) or greater, then the physician or equivalent thereof can diagnose a patient or subject has RA or ERA.

The kit can further comprise an insert that instructs how to adjust the therapeutically effective amount of anti-SR-A neutralizing antibodies to be administered to the subject based on the measurement of sSR-A and/or SR-A in the fluid biological sample.

One method to diagnose a patient having RA is identified above. That above-identified method is summarized as follows: (a) withdrawing or obtaining serum or a serum sample from a subject; (b) measuring a soluble macrophage scavenger receptor A (sSR-A) and/or a macrophage scavenger receptor A (SR-A) level in the serum or serum sample; and (c) diagnosing the subject has RA when the sSR-A and/or SR-A measurement is 1.7024 ng/ml or greater, and with greater certainty when the measurement of sSR-A and/or SR-A from the liquid biological sample is 5 ng/ml (or 7 ng/ml with even more certainty) or greater. Alternative methods to diagnose a patient having RA includes using the conventional cyclic citrullinated peptide (CCP) antibody ELISA kit that is publicly available. The former method is desirable because then the practitioner that administers the anti-SR-A neutralizing antibodies to the patient (or subject) having RA knows the level of sSR-A and/or SR-A in the patient (or subject) and can adjust the amount of anti-SR-A neutralizing antibodies administered to the patient (or subject) in accordance with the level of sSR-A and/or SR-A in the patient (or subject).

And the anti-SR-A neutralizing antibodies are selected from the group consisting of macrophage scavenger receptor A neutralizing polyclonal antibody (pAb), anti-human monoclonal antibody (mAb), pAb modified by galactosylation, mAb modified by galactosylation, pAb modified by sialylation, mAb modified by sialylation, and combinations thereof. The therapeutically effective amount of anti-SR-A neutralizing antibodies blockades or blocks the SR-A and sSR-A, and inhibits osteoclast differentiation and bone absorption in subjects experiencing PA. The therapeutically effective amount of anti-SR-A neutralizing antibodies are designed to treat certain effects of RA. Those certain effects are bone destruction in subjects experiencing RA, osteoclast differentiation and bone absorption in subjects experiencing RA, osteoclast differentiation in arthritis in subjects experiencing RA, and combinations thereof. The therapeutically effective amount of anti-SR-A neutralizing antibodies can also contain small-molecule inhibitors of SR-A. Those small-molecule inhibitors of SR-A are selected from the group consisting of tannic acid, rhein, and combinations thereof. The therapeutically effective amount of anti-SR-A neutralizing antibodies can also have inhibitory peptides of SR-A.

In addition, SR-A neutralizing antibody down-regulated the expression of osteocalcin- (OC) specific genes such as tartrate-resistant acid phosphatase (TRAP), cathepsin K (CTSK), and matrix metalloproteinase-9 (MMP-9), and attenuated the production of inflammation cytokine interleukin-6 (IL-6) and bone remodeling biomarker C-terminal telopeptide. Therefore, SR-A neutralizing antibodies provide a promising strategy for RA therapy especially in dampening bone destruction.

Ideal treatment in RA aims for both reductions of chronic inflammation and structural protection of the impaired bones and joints. However, current therapeutic regimens hardly achieve this perfect goal. Clinical observations indicated that compared with biologic agents, cDMARDs such as methotrexate (MTX), sulfasalazine (SSZ), leflunomide (LEF), and hydroxychloroquine (HCQ) display minimal protective effect on bone destruction in RA. As a bDMARDs, tumor necrosis factor (TNF)-blocking reagents, such as infliximab, could virtually arrest the progression of bone destruction in RA. Emerging studies have provided evidence that TNF-α may potentiate RANKL-induced OC differentiation, and impair the function of Treg cells which are potential suppressors of OC formation. Moreover, Abatacept, which can mimic the effect of cytotoxic T-lymphocyte-associated antigen 4 (CTLA4), could directly target cell surface receptor CD80/CD86 on precursor cells of OCs. tsDMARDs such as Janus-associated kinase (JAK) inhibitor were also reported to directly affect osteoclastogenesis. But these existing b/tsDMARDs are far from perfect due to the potential adverse effects, such as the higher risk of reactivation of latent tuberculosis and other serious opportunistic infections, malignancy, and the inadequate response in a proportion of patients.

The data shows that SR-A neutralizing antibodies significantly inhibit OC differentiation and bone destruction in patients with RA and CIA mice in vivo and in vitro. Given the anti-inflammatory effects of blocking SR-A in CIA mice as proved in our previous study, SR-A neutralizing antibodies provide a latent and supplementary strategy for RA therapy with dual protective functions. This partially explained the fast effect of SR-A neutralizing antibodies administration against osteoclastogenesis observed.

Currently, there are some commercially available SR-A neutralizing antibodies. For example and not limited, there are two SR-A neutralizing antibodies from R&D Systems were used: anti-mouse SR-A neutralizing polyclonal antibody (pAb) and anti-human monoclonal antibody (mAb). Novel anti-mouse SR-A mAbs for neutralization were successfully used for therapeutics. Moreover, anti-human SR-A neutralizing antibodies targeting the predominant epitopes, especially the pathogenic domain of soluble SR-A, require further development and review, since the results indicate that soluble SR-A and membrane-bound SR-A demonstrate different functions. In addition, further modification of SR-A neutralizing antibodies, such as galactosylation and sialylation enhance the anti-inflammatory activity, as well as partially or fully humanization that minimizes the immunogenicity, and further facilitate their clinical application.

Besides SR-A neutralizing antibodies, several other regents targeting SR-A, including small-molecule inhibitors (SMIs) of SR-A and inhibitory peptides, were developed for RA bone destruction treatment. Currently, tannic acid, rhein, and combinations thereof were identified as SMIs of SR-A. However, tannic acid revealed the ability to ameliorate inflammation and joint damage in arthritis mice. Moreover, through phage-displayed peptide library, a novel peptide antagonist of SR-A, PP1, was selected to target the carriers to atherosclerotic aortic artery lesions. Its effect against RA bone destruction remains unclear. Due to their unique mechanism of action and the simultaneous effect on multiple mediators, screening for more specific and competent SMIs and inhibitory peptides offer promising and supplementary strategies for the treatment of RA inflammation and bone destruction.

This data reveals the SR-A neutralizing antibodies inhibit OCs differentiation both in patients with RA and CIA mice, protecting against bone destruction. Developing more specific and optimal SR-A monoclonal antibodies and designing SMIs or inhibitory peptides will provide novel therapeutic strategies for treating bone destruction in RA.

While this disclosure has described certain embodiments and generally associated methods, alterations and permutations of these embodiments and methods will be apparent to those skilled in the art. Accordingly, the above description of example embodiments does not define or constrain this disclosure. Other changes, substitutions, and alterations are also possible without departing from the spirit and scope of this disclosure, as defined by the following claims.

1. A composition of a therapeutically effective amount of anti-macrophage scavenger receptor A neutralizing antibodies (anti-SR-A neutralizing antibodies) for treating effects of rheumatoid arthritis (RA) or early-stage rheumatoid arthritis (ERA) in a subject in need thereof, comprising
(a) measuring an amount of soluble macrophage scavenger receptor A (sSR-A) and/or a macrophage scavenger receptor A (SR-A) from a liquid biological sample obtained from the subject;
(b) diagnosing the subject has RA and/or ERA when the sSR-A and/or SR-A measurement is 1.7024 ng/ml or greater; and
(c) administering the therapeutically effective amount of anti-SR-A neutralizing antibodies to the RA and/or ERA diagnosed subject.

2. The composition of claim 1 wherein the anti-SR-A neutralizing antibodies are selected from the group consisting of macrophage scavenger receptor A neutralizing polyclonal antibody (pAb), anti-human monoclonal antibody (mAb), pAb modified by galactosylation, mAb modified by galactosylation, pAb modified by sialylation, mAb modified by sialylation, and combinations thereof.

3. The composition of claim 1 wherein the therapeutically effective amount of anti-SR-A neutralizing antibodies is adjusted in view the measurement of sSR-A and/or SR-A in the liquid biological sample; and ranges from:
(i) 0.1 mg/day to 10 mg/day;
(ii) 0.1 mg/every other day to 10 mg/every other day;
(iii) 0.1 mg/once a week to 10 mg/once a week;
(iv) 0.1 mg/once a month to 10 mg/once a month; and
(v) combinations thereof.

4. The composition of claim 1 wherein the therapeutically effective amount of anti-SR-A neutralizing antibodies to be administered to the subject is adjusted in view of the measurement of sSR-A and/or SR-A in the liquid biological sample.

5. The composition of claim 1 wherein the therapeutically effective amount of anti-SR-A neutralizing antibodies blockades the SR-A and sSR-A, and inhibits osteoclast differentiation and bone absorption in subjects.

6. The composition of claim 1 wherein the therapeutically effective amount of anti-SR-A neutralizing antibodies further comprises small-molecule inhibitors of SR-A.

7. The composition of claim 6 wherein the small-molecule inhibitors of SR-A are selected from the group consisting of tannic acid, rhein, and combinations thereof.

8. The composition of claim 1 wherein the therapeutically effective amount of anti-SR-A neutralizing antibodies further comprises inhibitory peptides of SR-A.

9. The composition of claim 1 wherein the administering the therapeutically effective amount of anti-SR-A neutralizing antibodies to the subject (a) down-regulates the expression of osteocalcin-specific genes such as tartrate-resistant acid phosphatase, cathepsin K, and matrix metalloproteinase-9, and (b) attenuates the production of (i) inflammation cytokine interleukin-6 and (ii) bone remodeling biomarker C-terminal telopeptide.

10. A method of using a therapeutically effective amount of anti-macrophage scavenger receptor A neutralizing antibodies (anti-SR-A neutralizing antibodies) for treating effects of rheumatoid arthritis (PA) or early-stage rheumatoid arthritis (EPA) in a subject in need thereof, comprising
administering the therapeutically effective amount of anti-SR-A neutralizing antibodies to the subject;
wherein the therapeutically effective amount ranges from:
(i) 0.1 mg/day to 10 mg/day,
(ii) 0.1 mg/every other day to 10 mg/every other day;
(iii) 0.1 mg/once a week to 10 mg/once a week;
(iv) 0.1 mg/once a month to 10 mg/once a month; and
(v) combinations thereof.

11. The method of claim 10 wherein the anti-SR-A neutralizing antibodies are selected from the group consisting of macrophage scavenger receptor A neutralizing polyclonal antibody (pAb), anti-human monoclonal antibody (mAb), pAb modified by galactosylation, mAb modified by galactosylation, pAb modified by sialylation, mAb modified by sialylation, and combinations thereof.

12. The method of claim 10 wherein the therapeutically effective amount of anti-SR-A neutralizing antibodies blockades the SR-A and sSR-A, and inhibits osteoclast differentiation and bone absorption in subjects.

13. The method of claim 10 wherein the therapeutically effective amount of anti-SR-A neutralizing antibodies further comprises small-molecule inhibitors of SR-A.

14. The method of claim 13 wherein the small-molecule inhibitors of SR-A are selected from the group consisting of tannic acid, rhein, and combinations thereof.

15. The method of claim 10 wherein the therapeutically effective amount of anti-SR-A neutralizing antibodies further comprises inhibitory peptides of SR-A.

16. The method of claim 10 wherein the treated effects of RA are bone destruction in subjects, osteoclast differentiation and bone absorption in subjects, osteoclast differentiation in arthritis, and combinations thereof.

17. The method of claim 10 wherein prior to administering the therapeutically effective amount of anti-SR-A neutralizing antibodies to the subject, the steps comprise:
withdrawing or obtaining a liquid biological sample from the subject wherein the liquid biological sample is selected from the group consisting of serum, plasma, synovial fluid, and combinations thereof;
measuring a soluble macrophage scavenger receptor A (sSR-A) and/or a macrophage scavenger receptor A (SR-A) level in the liquid biological sample;
diagnosing the subject has RA and/or ERA when the sSR-A and/or SR-A measurement in the liquid biological sample is 1.7024 ng/ml or greater.

18. The method of claim 17 further comprising the step of adjusting the therapeutically effective amount of anti-SR-A neutralizing antibodies to be administered to the subject in view of the measurement of sSR-A and/or SR-A in the liquid biological sample.

19. The method of claim 17 wherein the diagnosing the subject has RA and/or ERA occurs when the sSR-A and/or SR-A measurement in the liquid biological sample is greater than 5 ng/ml.

20. The method of claim 10 wherein administering the therapeutically effective amount of anti-SR-A neutralizing antibodies to the subject (a) down-regulates the expression of osteocalcin-specific genes such as tartrate-resistant acid phosphatase, cathepsin K, and matrix metalloproteinase-9, and (b) attenuates the production of (i) inflammation cytokine interleukin-6 and (ii) bone remodeling biomarker C-terminal telopeptide.

21. A kit comprising a container having at least a therapeutically effective amount of anti-SR-A neutralizing antibodies to be administered to a subject having a diagnosis of rheumatoid arthritis (PA) and/or early-stage rheumatoid arthritis (EPA); wherein the therapeutically effective amount ranges from:
(i) 0.1 mg/day to 10 mg/day,
(ii) 0.1 mg/every other day to 10 mg/every other day;
(iii) 0.1 mg/once a week to 10 mg/once a week;
(iv) 0.1 mg/once a month to 10 mg/once a month; and
(v) combinations thereof.

22. The kit of claim 21 wherein the container further has
a syringe to withdraw or obtain a liquid biological sample from the subject, the liquid biological sample is selected from the group consisting of serum, plasma, synovial fluid, and combinations thereof;
an immunoassay plate, after the syringe collects the liquid biological sample then (a) the immunoassay plate receives the liquid biological sample, (b) the liquid biological sample in the immunoassay plate is measured to quantify an amount of soluble macrophage scavenger receptor A (sSR-A) and/or macrophage scavenger receptor A (SR-A) level contained in the liquid biological sample, and when the measurement of sSR-A and/or SR-A from the liquid biological sample is 1.7024 ng/ml or greater, then the subject is diagnosed with RA and/or ERA.

23. The kit of claim 22 further comprising an insert that instructs how to adjust the therapeutically effective amount of anti-SR-A neutralizing antibodies to be administered to the subject based on the measurement of sSR-A and/or SR-A from the fluid biological sample.
